(19) United States
(12) Patent Application Publication
(10) Pub. No.: US 2015/0252409 A1
(43) Pub. Date: Sep. 10, 2015

(54) SYSTEMS GENETICS NETWORK REGULATORS AS DRUG TARGETS

(71) Applicant: Varigenix, Inc., Memphis, TN (US)

(72) Inventors: Robert E. Scott, Memphis, TN (US); Robert W. Williams, Memphis, TN (US)

(73) Assignee: Varigenix, Inc., Memphis, TN (US)

(21) Appl. No.: 14/198,135

(22) Filed: Mar. 5, 2014

(51) Int. Cl. C12Q 1/68

(52) U.S. Cl. CPC C12Q 1/6809

(57) ABSTRACT

The present invention provides for methods, processes and platforms to validate systems genetics networks to define their genetic regulators and to optimize translational applicability to humans for drug development. These systems genetics networks are sets of genes with a common function that demonstrate covariate expression that is genetically modulated by linked function network regulators (LFNRs) which comprise eQTLs in animals and GWAS SNPs in humans. LFNRs represent a new class of targets to identify drugs to prevent, ameliorate, and/or treat human diseases. LFNRs for the cell cycle-mitosis network have potential to be especially useful for anti-cancer therapies. The present invention provides for a drug that targets a specific LFNR for the cell cycle-mitosis network in Caucasian male liver to prevent the development of hepatocellular carcinoma in high risk patient populations.

### CROSS REFERENCE TO RELATED APPLICATIONS

This application claims priority under 35 U.S.C. 119(e) of U.S. Provisional Application Ser. No. 61/631,449, filed Jan. 5, 2012, the entire contents of which applications are hereby incorporated by reference in their entirety for any purpose.

### BACKGROUND OF THE INVENTION

1. Field of the Invention

The field of this invention relates to methods, processes and platforms for use to validate systems genetics networks of genes that share a common function and their genetic regulators that translate to humans as disease specific drug targets for drug discovery.

2. Description of the Related Art

Eukaryotic cell division proceeds through a highly regulated event, i.e. the cell cycle, comprising consecutive phases termed G1, S, G2 and M (mitosis). Disruption of the cell cycle or of cell cycle control mechanisms can result in cellular abnormalities or disease states, such as cancer. The dysregulation of cell cycle control can result from both genetic and epigenetic changes.

The transition of normal cells into precancerous and cancer cells involves multiple steps that typically occur over a period of many years. The key elements of carcinogenesis involve the sequential accumulation of mutations that activate oncogenes and disrupt cancer suppressor genes combined with multiple rounds of clonal selection and clonal evolution. Transient and stable epigenetic events also facilitate the development of cancer. Normal dividing cells are subject to a number of control mechanisms, known as cell-cycle checkpoints that can involve all four phases of the cell cycle (G1, S G2, M). Defects in one or more of these checkpoints are common during the process of carcinogenesis. An understanding of cell-cycle progression and cell-cycle control is therefore of significance in defining the molecular mechanisms that underlie carcinogenesis. From the perspective of genetics and systems genetics, each of these aspects of normal and aberrant proliferation control represent traits. Therefore the normal cell cycle and its components represent traits and aberrations in the cell cycle that occur during carcinogenesis and in cancers also represent distinct traits.

Genetics has been used in the field of trait analysis in order to identify the genes that regulate or modulate such traits. Key developments that has made it possible to study these traits in large populations of individuals required for systems genetics analysis have been: 1) the development of large collections of molecular or genetic markers in mice, rats, humans and other species/organisms, which can be used to construct detailed genetic maps and 2) bioinformatics and computer technologies that make it possible to evaluate derived datasets, i.e., the open source GeneNetwork data analysis system (www.genenetwork.org).

Systems genetics or “network genetics” is an emerging new branch of genetics that aims to understand complex causal networks of interactions at multiple levels of biological organization. To put this in a simple context: whereas Mendelian genetics can be defined as the search for linkage between a single trait and a single gene variant (1 to 1); complex trait analysis can be defined as the search for linkage between a trait and a set of gene variants [quantitative trait loci (QTLs) and associated quantitative trait genes (QTGs) with one to many environmental cofactors].

Systems genetics technologies employ quantitative trait locus (QTL) mapping, such as interval mapping, simple interval mapping, composite interval mapping, multiple and composite interval mapping. QTL mapping methodologies provide statistical analysis of the association between phenotypes and genotypes for the purpose of understanding and dissecting the regions of a genome that modulate traits and complex traits.

Interval Mapping is a method of using statistical tests of association between trait values and the genotypes of marker loci through the genome. A significant association is interpreted as indicating the presence of a QTL linked to the marker that causes the association.

Simple interval mapping is a method for evaluating the association between the trait values and the known or imputed genotype at chromosomal positions at or between sets of adjacent genotyped markers.

Composite interval mapping also evaluates the association at analysis points across chromosomal positions. However, analysis also includes a computation method to control for the effect of one or more genotype markers elsewhere in the genome. These markers, also called background markers, have previously been shown to be associated with the trait and therefore are each presumably close to another QTL (a background QTL).

Multiple interval mapping uses multiple marker intervals simultaneously to fit multiple putative QTL directly in the model for mapping QTL.

A QTL is a chromosome region that contains one or more sequence variants that modulates the distribution of a variable trait measured in a sample of genetically diverse individuals from an interbreeding population. Variation in a quantitative trait may be generated by a single QTL with the addition of some environmental noise. Variation may be oligogenic and be modulated by a few independently segregating QTLs. In many cases however, variation in a trait will be polygenic and influenced by large number of QTLs distributed on many chromosomes. Environment, technique, experimental design and a host of other factors also affect the apparent distribution of a trait. Therefore, most quantitative traits are the product of complex interactions of genetic factors, developmental and epigenetics factors, environmental variables, and measurement characteristics.

The goal of identifying all such regions that are associated with a specific complex phenotype can be difficult to accomplish because of the existence of multiple QTLs, the possible epistasis or interactions between QTLs, as well as many additional sources of variation that can be difficult to model and detect.

QTLs may be used to identify candidate genes underlying a trait, i.e., quantitative trait genes (QTGs). QTLs can be associated with large numbers of potential QTGs that typically range from 50 to several 100, therefore making it difficult to define which candidate QTG(s) might serve a modulatory role for the trait of interest.

In recent years, QTL analyses have been combined with gene expression profiling, i.e., quantitative RNA analysis using microarrays, RNA sequencing, or quantitative polymerase chain reaction analysis. Such expression QTLs (eQTLs) can include genes whose expression is influenced by either cis-acting (close to the parent gene of the RNA types) or trans-acting (not close to the parent gene of the RNA type) control systems.

Historically, the availability of adequately dense markers (genotypes) has been the limiting step for QTL analysis. However, high-throughput technologies and genomics have begun to overcome this bather. Thus, the remaining limitations in QTL analysis are now predominantly at the level of defining QTGs

The regulation of systems genetics network characteristics in animal populations, such as recombinant inbred BXD mice, involves QTLs and QTGs. In humans, genome wide association studies (GWAS), are being used to define similar regulators or modulators of network biology, i.e., GWAS SNPs.

In genetic epidemiology, a genome-wide association study (GWA study, or GWAS), also known as whole genome association study (WGA study, or WGAS), is an examination of many common genetic variants in different individuals to see if any variant is associated with a trait. GWAS typically focus on associations between single-nucleotide polymorphisms (SNPs) and traits such as those of major diseases.

These studies normally compare the DNA of two groups of participants: people with the disease (cases) and similar people without the disease (controls). Each person gives a sample of DNA, from which millions of genetic variants are typed using SNP arrays. If one type of the variant (one allele) is more frequent in people with the disease, the SNP is then the to be “associated” with the disease. The associated SNPs are then considered to mark a region of the human genome that influences the risk of disease.

Genome-wide association studies have become a powerful tool. However, genome-wide association studies by themselves do not provide complete insight into the mechanisms through which genetic variation drives phenotypic variation.

Considering the large number of traits profiled in a single microarray hybridization combined with the many other endpoints measured, all of which are performed across a population of individuals, data density is an inherent feature of systems genetics studies. Therefore, it is a discipline that is interdisciplinary by nature, requiring extensive collaborations among biologists, statisticians, and computational biologists.

Relations among traits are extracted using a variety of computational approaches, all of which begin with some measure of pairwise correlation between traits. Typically, the process of assembling phenotypic networks begins at the level of gene expression, where sets of transcripts with similar patterns of expression across the population are extracted from large-scale gene expression data. The rationale behind co-expression networks is that genes encoding RNA and proteins that function in the same pathway will display coordinated expression across the population to the extent that they are regulated at the level of mRNA abundance. Progressing from the very large correlation matrices created from microarray data to identification of smaller sets of co-expressed genes requires some level of thresholding, i.e., selecting a correlation value above which relationships are considered meaningful. After thresholding, a variety of methods are used to identify putative co-expression networks and link them to higher order physiological traits. Graph algorithms, which represent traits as nodes and the correlations between transcripts as edges, are widely used to represent the interactions between genes after thresholding. Graphs can be weighted, in which edges retain information about the magnitude of correlation between transcripts, or unweighted, with all edges treated equally. As an example, GeneNetwork (www.genenetwork.org) employs these and additional technologies to make possible the advanced analysis of systems genetics datasets.

Network-based approaches that are central to systems genetics are also ideal for determining mechanisms through which environmental variables can affect a biological system across a population.

Many variables and complexities exist in the experimental systems that are commonly used to identify systems genetics expression networks using microarray technologies and the genetic regulators/modulators that influence their characteristics. These include: 1) variability in the preparation of batches of cells or tissues from different genetic variation panels, 2) variation in the preparation of the RNA for microarray analysis from such panels, 3) variation in microchip technology and microarray analysis procedures including data normalization procedures, 4) variability in the characteristics of similar databases prepared in different laboratories by different investigators, 5) complexity in the identification of the loci of genetic regulators/modulators for specific networks, such as, eQTLs, and 6) complexity in the identification of actual genetic regulators or modulators for specific networks, such as, eQTGs, due to limitations in sample size and other parameters that yield large numbers of candidate regulatory/modulatory genes. All of these limitations and complexities can make systems genetics studies difficult to interpret and reproduce.

A major challenge currently exists concerning the identification of regulatory or modulatory genes (QTGs) for specific systems genetics networks. The challenge in using today's technology is that regulatory or modulatory regions of DNA typically encompass too many candidate genes to definitively identify functional network regulators or modulators.

Throughout the remainder of this document the term genetic regulator is used rather than genetic regulator or modulated or related terms. As such the term genetic regulator is to be understood to include functions that regulate or modulate the expression of network gene sets to different degrees that can vary from partial to complete and all other variations.

The inventors of the present invention have now invented a process to optimally define biological networks and their regulators. An important aspect of the present invention is the multiple criteria validation (MCV) process that is used to validate a biological network of interest in many different species, different strains, different tissues, different cells and/or different sexes using many databases developed from such different genetic variation assay panels. Based on the characteristics of the validated networks, the genetic regulators for the network can then be studied from a variety of perspectives. That make it possible to identify eQTLs in the different datasets and thereby to identify eQTGs for the network in multiple situations so as to define those eQTGs that have a linked function in multiple situations and wherein those linked function eQTGs have a high probability of serving a regulatory function for the network characteristics. These designated linked-function network regulators (LFNRs) can be one or more eQTGs in animals whereas in humans they can be one or more GWAS SNPs. The present invention has focused on using the MCV process to validate a special systems genetics network designated the cell cycle-mitosis network. This systems genetics network is of high clinical significance for human disease, especially cancer, because of the importance of cell cycle and mitosis lesions that occur during carcinogenesis and in cancers. The inventors have found that the cell cycle-mitosis network and the LFNR principle that they defined in systems genetics studies on recombinant inbred strains of mice and rats, translates with very high relevance to the cell cycle-mitosis network in human populations, specifically involving human liver. Thereby a cell cycle-mitosis network has been defined in male human liver and female human liver and a select few significant specific GWAS SNPs for such networks have been defined in each sex. The inventors have also established that the most significant LFNR (GWAS SNP) for the cell cycle-mitosis network in Caucasian male human livers, has a high potential to serve as a liver cancer prevention drug target and that an existing class of clinical drugs is known to inhibit the activity of that LFNR and thereby serve as a candidate liver cancer prevention drug for use in Caucasian males at high risk of developing liver cancer.

Discussion or citation of a reference herein will not be construed as an admission that such reference is prior art to the present invention.

### SUMMARY OF THE INVENTION

The present invention provides an improvement over the art by uniquely combining methods, processes and platforms to validate the preclinical discovery of systems genetics networks and their genetic regulators with human translation applicability for drug development such as when using gene expression profiling approaches to define networks of covariate genes associated with complex traits, such as the cell cycle-mitosis network and its functional regulators, which can then serve as a new class of drug targets, such as for cancer prevention and cancer therapy.

In one embodiment, a multiple criteria validation (MCV) process is used to assure the reproducibility of systems genetics covariate gene expression networks with functional significance that show species, sex and tissue specific characteristics and thereby to define such a systems genetics network as a worthwhile focus of continued analysis to define the genetic regulators of the network that can be used a targets for drug develop that translates to humans.

In another embodiment, the MCV process provides the validation necessary for the subsequent development of the LFNR platform that serves to identify Linked-Function Network Regulators that can influence the characteristics of systems genetics covariate gene expression networks, such as the cell cycle-mitosis network.

In another embodiment, the invention provides for cell cycle-mitosis networks and their LFNRs (eQTGs) in interbreeding non-human animal populations, such as recombinant inbred mice and rats, with species, strain, sex and tissue specificity. In another embodiment, the invention provides for the use of cell cycle-mitosis networks and their LFNRs derived from animal studies to predict the characteristic of the cell cycle-mitosis network and their LFNRs (GWAS SNPs) in humans with one or more of race, sex, and tissue specificities.

The present invention also provides that the multiple criteria process to validate a systems genetics network of genes that have a common function comprises: selecting a candidate network comprising covariate expressed genes that have a common function identified as associated with a gene of interest in a test population; and determining if the identified candidate systems genetics network show covariate expression of network genes in a population data set selected from the group consisting of: two or more tissue or cell types; two or more data sets developed by different laboratories or different investigators or both; two or more different microarray platforms; two or more different animal species or strains; and two or more different microarray data normalization systems; wherein the identified candidate systems genetics network is validated if it is determined that the network of covariate expressed genes with a common function are identified as having correlation coefficients greater than or equal to 0.5 or higher in two or more of the test populations.

In one embodiment, the process further compromises the step (c) determining that the identified candidate systems genetics network has one or more suggestive or significant eQTLs in one or more test populations by using one or more systems genetics bioinformatics tool.

In another embodiment, the process further comprises the step (d) determining that the identified candidate systems genetics network exists substantially more in tissues or cells that physiologically express the function of the identified network than in tissues or cells that do not express the function or express the function to a lesser degree or extent. In another embodiment, the step of validating the candidate network is determined by a process comprising (i) using one or more microarray-based gene expression bioinformatics data sets; and (ii) analyzing the bioinformatics outcomes to validate the candidate network of interest; wherein each bioinformatics data set is made up of genetically diverse panel of specimens from large populations of genotypes.

In another embodiment, the gene expression data set defines gene expression covariates for a specific genetic variation panel of cells, tissues or animals. In another embodiment, one or more microarray-based gene expression data sets is analyzed by using bioinformatics tools from the group consisting of GeneNetwork, BisoGenet, Cytoscape, VisANT, Osprey and Biological Networks. In another embodiment, one or more gene ontology analysis system is used to define expression covariate gene sets that share a common function in such a population.

In another embodiment, the system is a computer-based system comprising a computer readable storage medium and a computer program mechanism embedded therein, the computer program mechanism comprising a construction module for constructing a gene network, comprising: (i) instructions for converting one or more types of biological data respectively into a representation of values; (ii) instructions for using each representation of values as a probability in a computational model to construct the gene network.

In another embodiment, a plurality of transcripts for the gene of interest is used to identify and study specific candidate networks in each data set and wherein transcripts for the selected genes that comprise the specific candidate network can also be used to identify specific candidate networks in the data set.

The present invention also provides for methods for identifying the linked function network regulator of a systems genetics network of interest comprising screening a plurality of eQTLs from multiple populations and identifying a linked function shared by the candidate eQTGs in each population; wherein the eQTGs identified as having a linked function are designated as candidate linked function network regulators (LFNRs) for the network.

In one embodiment, the linked function network regulator is a gene product with a function linked with the network regulated by the linked function network regulator. In another embodiment, the linked function network regulator is not a gene product linked to the network regulated by the linked function network regulator. In another embodiment, the candidate eQTGs associated with the eQTLs of the network of interest in various populations are analyzed using bioinformatics tools. In another embodiment, the eQTLs for the network of interest contain a distinct composition of genes with a linked function in a plurality of populations selected from the group consisting of species, strains, tissues, cell types and sexes. The identified eQTGs may act in cis or in trans.

In another embodiment, the method includes the further step of defining the candidate eQTGs associated with eQTLs for a specific network by identifying the eQTGs in multiple populations and where all cis and/or trans candidate eQTGs are analyzed for each of populations to identify a linked function shared by the candidate eQTGs in each population. In another embodiment, a subset of the candidate cis and/or trans eQTGs is identified as having a linked function that is shared with each population and wherein the subset genes identified are designated as the linked function network regulators for the network.

In another embodiment, the identified LFNRs for a specific network are defined from datasets of a large animal population and wherein information concerning the animal LFNR characteristics of the specific network is used to predict LFNR characteristics in human populations for the corresponding human network.

The present invention provides for a data set of genes that comprise a network that share a common cell cycle and/or mitosis function whose expression is covariate and whose function is regulated by a linked function network regulator.

In one embodiment, the covariate expressed genes have correlation coefficients greater than or equal to 0.5 in a population selected form the group consisting of different species, strains, sexes, tissues and cells. In another embodiment, the network exists in a plurality of tissues and cells having proliferative potential. In another embodiment, the network exists in at least 10 tissues having proliferative potential. In another embodiment, the tissues are selected from the group consisting of liver, lung, spleen, kidney hematopoietic stem cells, thymus, cartilage, the eye, adipose tissue and lymphocytes. In another embodiment, the dataset comprises a subset of less than 775 genes. In another embodiment, the dataset comprises a subset of less than 166 genes. In another embodiment, the dataset comprises a subset of genes in a range from about 25 to about 60 genes.

In one embodiment, the covariate expressed genes are one or more of Cdc20, Aurka, Nuf2, Cenpf, Nek2, Nusap1, Tpx2, Ube2c, Ccna2, Cenpe, Cdca8, Prc1, Mki67, Ccnb2, Aurkb, Spag5, Birc5, Cenph, Racgap1, Sgol1, Kif20a, Cdca5, Kntc1, Plk4, Cenpa, Plk1, Cdc2a, Ncapg, Incenp, Top2a, Npdc1, Ncaph, Ktcn2, Cdca3, Cdca1 and Ccnb1, Cdc2, Cdc25c, Mphosh1, Uhrf1, Scyl3, Pbk, Shcbp1, Pkmyt1, Exo1, Gtsel, Stmn1, Chek, Cdc451, Cenpt, Mad2l1, Zwilch, Smc2, Anin, Cdc42, Ncapd2, Bub1b, Ttk, Anapc5, Cdca4, Aspm, Kif22, Cdc1, Ckap21, Zwint, Wee1, Cdk2, Pstpip1, Cdt1, Fbxo5, Sertad2, Dbf4, Lig1, Smc2l1, Spag1, Cenpp, Solt, Fshprh1, Ccnf, Cks2, Brrn1, Cdc91l1, Ereg, cks1b, Pardbg, Psen, Htatip2, Katna1, Rbbp8, Spin, Camk2d, Tgfb2, Pola, Nfatc1, Trp53bp1, Tubb5, Ndc1, Ncapd3, Spc24, Numa1, Cenpb, Cenpm, Smc4, Cenpi, Smc2, Cep55, Tipin, Ndc80, Kifc1, Cdc123, Cdca2, Spc25, Kif23, Ccna2, Stmn1, Dlgap5, Kif4a, Timeless, Aurkc, Cdc25a, Cdc6, Espl1, Kif2c, Cenpn, Cdca3, Brac2, Fzr1, Tubg1, Ckap5, Numa1, Nudc, Scyl3, Tacc3, Shcbp1, Bub1, Sgol2, Cdc25b, Mcm2, Mcm4, Mcm5, Mcm7, Myc, Spc24, Kif24, Kif11, Ndc80, Epr1, Ttk, Mybl2, Plk1, Kif14, Cdkn2c, E2f2, Aurkaps1, Pttg1, Cit, Mast1, Melk, Psrc1, Casc5, Mcm6, Chaf1, Gmnm, Cdc7, Spbc25, Chek1.

In one embodiment, a plurality of transcripts for the gene of interest is used to identify candidate networks in each data set and wherein transcripts for the selected genes that comprise the specific candidate network are used to identify specific candidate networks in each data set. In another embodiment, the eQTLs are identified for the cell cycle-mitosis network in a plurality of tissues and cells of different species, strains, sexes and wherein the eQTLs with associated eQTGs are used to identify a linked function network regulator for the cell cycle-mitosis network in each situation. In another embodiment, the representative eQTLs are selected from the group consisting of BXD male mouse liver chromosome 2 Mb 100 to 135, BHHBF2 male liver chromosome 11 Mb 102 to 116 and chromosome 17 Mb 12 to 28, BXD lung of combined sexes chromosome 9 Mb 110 to 125, BXD spleen of combined sexes chromosome 15 Mb 85 to 100, BHHBF2 male adipose tissue chromosome 4 Mb 45 to 70 and chromosome 6 Mb 35 to 50 and male adipose tissue chromosome 2 Mb 4 to 21 and chromosome 8 Mb 88 to 100.

In another embodiment, the set of candidate cis eQTGs includes BXD male liver genes Lmo2, Ltk, Mga, Sirm (Zfp106), Slca2, Mmrp19 (Apip), Ivd, Itpka, Rgap1 (1Racgap1), PLA2G4B Pla2g4b (Pa24b), Capn3, Cnndbp1 (Gcip), Catsper2, Mfap1, B2m, Sdh1 (Sdhb), Slc30a4, Cops2 (Alien), Mpped2, Fibin, Fam82a2, Gchfr, Tmem87a, Haus2 (Cep27) or Adal.

In another embodiment, the set of candidate cis eQTGs includes BHHBF2 male liver genes Prkar1a, Wtap, Pkmyt1, Ccnf, Tsc2, Acbd4 Kpna2, Helz, Cog1, Cd300a, Rnf157, St6gainc2, Syngr, Map3k4, Pnldc1, Acat2, Tceb2, Zfp598, Gfer, Tbl3, Traf7, Rps2, Hs3st6, Nubp2, Ift140, Telo, Gnptg, Wfikkn1, Decr2 or Tmem8.

In another embodiment, the set of candidate cis eQTGs includes BXD lung genes of combined sexes Rmbs3, Limd1, Clasp2, Champ (Mov10l1), Ifrd2, Ccdc72, Tmem7, Crtap, Glb1, Acaa1b, Acaa1, Rpl14, Sec22l3, Deb1, Nktr, Hig1, Ccbp2, Ccr1, Ccr2, Ccr5, Ulk4 or Tmem103.

In another embodiment, the set of candidate cis eQTGs includes BXD male spleen genes selected from the group consisting of Epas (Rapgef3), Ttll12, Arsa, Kif21a, Pp11r, Tmem106c, Senp1, Adcy6 and Accn2.

In another embodiment, the set of candidate cis eQTGs includes BHHBF2 male adipose tissue genes Hoxa2, Smc2, Tbxas1, Rab19, Ndufb2, Gstk1, Zfp467, Rarres2, Zfp775, Tmem176b, Gpnmb, Cdcc126, Mpp6, Dfna5h, Skap2, Hibadh, Plekha8, Gars, Mcart1, Txndc4, Ecm29, Gbg10, Bspry, Alad or Zfp618.

The article of claim 53, wherein the total set of candidate cis eQTGs includes BHHBF2 male adipose tissue genes Gadd45gip1, Usp38, Elmod2, Cd97, Asf1b, Trmt, Lul1, Rad23a, Farsia, Gcdh, Fbxw9, Vps35, Mmp2, Capns2, Pllp, Ciapin1, Gpr97, Gins3, Ndrg4, Usp6n1, Ptpla, Scl339a12, Armc3 or Lcn4.

In another embodiment, the candidate cis eQTGs for the cell cycle-mitosis network that share a linked function and thereby represent candidate LFNRs are selected from the group consisting of (i) BXD liver genes Mga, Ccndbp1, Mfap1, Cops2, Mpped2, and Haus2; (ii) BXD lung genes Rbms3, Clasp2, Champ, and Nktr; BXD spleen genes Epac and Senp1; (iii) BHHBF2 liver genes Wtap, Pkmyt1, Ccnf, Nubp2, Tsc2 and Gfer; and (iv) BHHBF2 adipose tissue genes Smc2, Hoxa2, Gadd45gip1, Asf1b, Ciapin1, Ndrg4 and Usp6n1.

In another embodiment, the linked function of the candidate LFNR (eQTG) is a cell cycle or mitosis function and the data set is a database tangibly embodied on a computer-readable medium. In another embodiment, the characteristics of the cell cycle-mitosis network and the LFNRs for the network in non-human animals provides a model for translation to humans as new drug targets for the prevention, amelioration or treatment of cancer and other human diseases.

The present invention further provides for a method for identifying human candidate cell cycle-mitosis networks and their linked function network regulators, the method including the steps of: selecting a human gene expression data set of interest representing a population of tissues or cells with significant genetic variation and analyzing the data set using a candidate gene of interest to identify cell cycle and/or mitosis genes whose expression is covariate; selecting a set of genes having cell cycle and/or mitosis function and designating that set of genes as a network.

In one embodiment, the data set comprises information based on studies in non-human animal populations having comparable genetic variation. In another embodiment, the human populations of one or more types of cells and/or tissues are selected based on one or more characteristic selected from the group consisting of race, sex, ethnicity, geography, age, and other identifiable population characteristics. In one embodiment, the human population-based data sets are obtained from at least 10, 100, 200, 300, 400, 500, 1,000, 2,000, 3,000, 4,000, or 5,000 or greater number of human subjects.

In another embodiment, the data sets used to screen for the cell cycle-mitosis network and for GWAS SNPs employ gene expression information obtained from whole genome expression arrays or from specially designed sets of gene expression arrays that related to the cell cycle and/or cancer. In another embodiment, the method includes identifying GWAS SNPs for the selected cell cycle-mitosis network genes in a plurality of human tissue or cell populations

In another embodiment, the tissues are selected from the group consisting of liver, lung, spleen, kidney, thymus, lymph nodes, vascular tissues, cartilage, bone, pancreas, the eye, adipose tissue, gastrointestinal tract, blood and bone marrow cells, lymphocytes endocrine tissues, reproductive tissues and selected neural tissues and wherein the tissues are normal, diseased, premalignant or cancerous.

In another embodiment, the GWAS SNP candidates having the highest significance and having a cell cycle or mitosis function are designated as candidate LNFRs. In another embodiment, the GWAS SNPs have a significance of 4.0−log P or greater. In another embodiment, the GWAS SNPs have a significance of 5.0−log P or greater. In another embodiment, the GWAS SNPs have a significance of 8.0−log P or greater.

In another embodiment, the GWAS SNP analysis for the cell cycle-mitosis network in the human specimens comprises use of GeneNetwork or comparable bioinformatics analysis tools. In another embodiment, the cell cycle-mitosis network and its LFNRs are defined for human Caucasian female and male liver tissues. In another embodiment, the LFNRs for the cell cycle-mitosis network provide for new drug targets for the prevention, amelioration or treatment of cancer and other human diseases.

The present invention also provides for a human Caucasian female liver data set of genes wherein the genes (a) exhibit have covariate gene expression and (b) share a common cell cycle and/or mitosis function that is regulated by a linked function network regulator (LFNR).

In one embodiment, the network comprises a plurality of covariate genes selected from the group consisting of Cdc20, Nusap1, Cdc14b, Foxn3, Lig1, Mcm10, Ccnf, Crebl2, Ccng1, Tbx2, Cdca2, Mybl2, Pip4r1, Ube2c, Kif2c, E2f2, Ncaph, Kifc1, Kif23, Ttk, Foxm1, Pttg2, Ccnb2, Plk1, Cdca8, Exo1, Orcgl, Cdca3, Cdca5, Orc1l, Cenph, Kif11, Aspm, Pttg1, Cep25b, Zwint, Aurkb, Ccnb1, Cenpa, and Hmmr genes.

In another embodiment, the network comprises Nusap1, Cdc14b, Foxn3, Lig1, Mcm10, Ccnf, Crebl2, Ccng1, Tbx2, Cdca2, Mybl2, Pip4r1, Ube2c, Kif2c, E2f2, Ncaph, Kifc1, Kif23, Ttk, Foxm1, Pttg2, Ccnb2, Plk1, Cdca8, Exo1, Orcgl, Cdca3, Cdca5, Orc1l, Cenph, Kif11, Aspm, Pttg1, Cep25b, Zwint, Aurkb, Ccnb1, Cenpa, and Hmmr genes.

In another embodiment, the covariate expressed genes of the human Caucasian female liver cell cycle-mitosis network have correlation coefficients greater than or equal to 0.5. In another embodiment, a plurality of transcripts for the gene of interest is used to identify the cell cycle-mitosis network in and wherein transcripts for the selected genes that comprise the network can also be used to identify the network. In another embodiment, GWAS SNPs are identified for the cell cycle-mitosis network wherein the GWAS SNPs that are associated with genes that have a function linked to the cell cycle and/or mitosis are designated as candidate linked function network regulators for the cell cycle-mitosis network.

In another embodiment, Caucasian female liver cell cycle-mitosis network genes containing GWAS SNPS that have a significance >8.0−log P are candidate linked function network regulators for the network. In another embodiment, genes selected from the group consisting of Astn2 and Tbx19 are candidate linked function network regulators for the cell cycle-mitosis network. In one embodiment, Astn2 is the candidate linked function network regulators for the cell cycle-mitosis network. In another embodiment, Tbx19 is the candidate linked function network regulators for the cell cycle-mitosis network.

In another embodiment, Caucasian female liver cell cycle-mitosis network genes containing GWAS SNPS that have a significance from 5.0 to 8.0−log P are candidate linked function network regulators for the network. In another embodiment, the genes selected from the group consisting of Cxad, Nrg1 and Prdm16 are candidate linked function network regulators for the cell cycle-mitosis network. In another embodiment, the gene Cxad is the candidate linked function network regulator for the cell cycle-mitosis network.

In another embodiment, Caucasian female liver cell cycle-mitosis network genes containing GWAS SNPS that have a significance from 4.0 to 5.0−log P are candidate linked function network regulators for the network. In another embodiment, the genes selected from the group consisting of Dapp1, Cenph, Cdk2ap1, Nell1 and Symd3 are candidate linked function network regulators for the cell cycle-mitosis network. In another embodiment, the various candidate LFNRs for the Caucasian female liver cell cycle-mitosis network represent candidate drug targets for prevention, amelioration, or treatment of cancer and other diseases. In another embodiment, the linked function is selected from the group consisting of cell cycle and mitosis functions and wherein the data set is a database tangibly embodied on a computer-readable medium.

The present invention also provides for a method of testing candidate drug targets comprising assessing the functional impact on the gene expression product for the candidate linked function network regulators and the characteristics of the cell cycle and mitosis functions during or following RNAi treatment using a RNAi for the specific LFNR of interest. In another embodiment, the method further comprises screening small molecule compound libraries to identify one or more compounds that impact the activity or expression of the gene product drug target.

The present invention also provides for a method for determining or measuring if a test compound or compounds or a putative drug composition(s) can modify or alter the physiology of a cell, comprising determining the gene expression of one or more candidate linked function network regulators for the cell cycle-mitosis network in a cell or cells of interest, and determining the gene expression of the same or equivalent cell or cells after: providing a test compound or compounds or a putative drug composition(s); providing a cell or cells; contacting the test compound or compounds or the putative drug composition(s) of (a) with the cell or cells of (b); and determining or measuring a difference or change in the gene expression of the cell or cells, wherein a difference or change in the gene expression signature of the cell or cells between step (i) and step (ii), or a difference or change in the gene expression signature of the cell or cells after contacting or culturing the cells or cells with the test compound or compounds or putative drug composition(s), identifies the test compound or compounds or putative drug composition(s) as a composition or drug that can modify or alter the physiology of the cell; wherein the gene expression signature of the cell or cells is determined by a method using a chip, a microassay, or a biochip.

The present invention also provides for an article comprising a human Caucasian male liver data set of genes wherein the genes (a) exhibit have covariate gene expression and (b) share a common cell cycle and/or mitosis function that is regulated by a linked function network regulator (LFNR). In one embodiment, the network comprises a plurality of covariate genes selected from the group consisting of Cdc20, Cdc123, Cdk2, Mybl2, Kif2c, Ube2c, Ccnf, Cdca2, Plk1, Ckap21, Pttg2, Cdca3, Pole, Lig1, Cdca8, Ncaph, Kifc1, Mcm10, Tbx2, Foxm1, Aspm, Kif23, Ccnb2 and Ttk. In another embodiment, the network comprises Cdc20, Cdc123, Cdk2, Mybl2, Kif2c, Ube2c, Ccnf, Cdca2, Plk1, Ckap21, Pttg2, Cdca3, Pole, Lig1, Cdca8, Ncaph, Kifc1, Mcm10, Tbx2, Foxm1, Aspm, Kif23, Ccnb2 and Ttk. In another embodiment, the covariate expressed genes of the human Caucasian male liver cell cycle-mitosis network have correlation coefficients greater than or equal to 0.5.

In another embodiment, the Caucasian male liver cell cycle-mitosis network genes containing GWAS SNPs that have a significance >8.0−log P are candidate linked function network regulators for the network. In another embodiment, the Aro1 is the candidate linked function network regulators for the cell cycle-mitosis network. In another embodiment, the Caucasian male liver cell cycle-mitosis network genes containing GWAS SNPs that have a significance from 5.0 to 8.0−log P are candidate linked function network regulators for the network. In another embodiment, the gene Angpt2 is the candidate linked function network regulator for the cell cycle-mitosis network. In another embodiment, Caucasian male liver cell cycle-mitosis network genes containing GWAS SNPS that have a significance from 4.0 to 5.0−log P are candidate linked function network regulators for the network. In another embodiment, genes selected from the group consisting of Wwc1, Npas3, Ptprg and Traf3ip1 are candidate linked function network regulators for the cell cycle-mitosis network. In another embodiment, the various candidate LFNRs for the Caucasian male liver cell cycle-mitosis network represent candidate drug targets for prevention, amelioration, or therapy of cancer and other diseases. In another embodiment, the various candidate LFNRs for the Caucasian male liver cell cycle-mitosis network represent candidate drug targets for prevention, amelioration, or treatment of cancer and other diseases. In another embodiment, the protein product of the Aro1 gene that represents the most significant candidate LFNR for the cell cycle-mitosis network in Caucasian male liver is a target for the aromatase inhibitor class of drugs that are currently used extensively for the treatment of human diseases.

The present invention also provides for a method of testing candidate drug targets comprising assessing the functional impact on the gene expression product for the candidate linked function network regulators and the characteristics of the cell cycle and mitosis functions during or following RNAi treatment using a RNAi for the specific LFNR of interest. The method further comprises screening small molecule compound libraries to identify one or more compounds that impact the activity or expression of the gene product drug target.

The present invention also provides for a method for determining or measuring if a test compound or compounds or a putative drug composition(s) can modify or alter the physiology of a cell, comprising: determining the gene expression of one or more candidate linked function network regulators for the cell cycle-mitosis network in a cell or cells of interest, and determining the gene expression of the same or equivalent cell or cells after: providing a test compound or compounds or a putative drug composition(s); providing a cell or cells; contacting the test compound or compounds or the putative drug composition(s) of (a) with the cell or cells of (b); and determining or measuring a difference or change in the gene expression of the cell or cells, wherein a difference or change in the gene expression signature of the cell or cells between step (i) and step (ii), or a difference or change in the gene expression signature of the cell or cells after contacting or culturing the cells or cells with the test compound or compounds or putative drug composition(s), identifies the test compound or compounds or putative drug composition(s) as a composition or drug that can modify or alter the physiology of the cell; wherein the gene expression signature of the cell or cells is determined by a method using a chip, a microassay, or a biochip.

In another embodiment, the LFNR for the cell cycle-mitosis network in the liver of Caucasian males is the aromatase gene Aro1 (CYP19A1).

In one embodiment, the present invention is directed to pharmaceutical compositions and methods of use for the prevention or reduction of incidence of liver cancer for aromatase inhibitor treatment in a human Caucasian male subject. In another embodiment, the subject is afflicted with chronic viral hepatitis, which may be with or without evolving cirrhosis.

The present invention provides for a method of treatment for preventing or reducing the incidence or severity of liver cancer in a Caucasian human male patient identified as being in need of such treatment comprising administering to the patient one or more doses of at least one aromatase inhibitor that targets the Aro1 gene product, either alone or in conjunction with another pharmaceutical agent, in an amount effective to prevent or reduce the incidence of liver cancer in the patient.

In one embodiment, the male Caucasian patient has chronic viral hepatitis with or without cirrhosis. In another embodiment, the liver cancer is hepatocellular carcinoma (HCC). In another embodiment, the aromatase inhibitor has a steroidal or non-steroidal chemical structure. In another embodiment, the at least one aromatase inhibitor is selected from reversible and non-reversible aromatase inhibitors.

In another embodiment, the at least one aromatase inhibitor is a third generation inhibitor selected from the group consisting of anastrozole, formestane, aminoglutethimide, fadrozole, letrozole, vorozole, exemestane and a pharmaceutically acceptable salts and derivatives thereof. In another embodiment, from 1 to 10 daily doses of the at least one aromatase inhibitor are administered. In another embodiment, at least one aromatase inhibitor is administered in a daily dose of from about 0.1 mg to about 50 mg. In another embodiment, at least one aromatase inhibitor is administered orally. In another embodiment, at least one aromatase inhibitor is a pharmaceutical composition comprising a therapeutically effective amount of an aromatase inhibitor and a pharmaceutically acceptable carrier.

In another embodiment, the pharmaceutical composition further comprises a therapeutically effective amount of an additional anti-cancer agent. In another embodiment, the male Caucasian patient is diagnosed as having a precancerous condition. In another embodiment, the male Caucasian patient has the disease of chronic viral hepatitis with or without cirrhosis that transforms into hepatocellular carcinoma at an annual rate of 3 to 8% dependent on the type of viral hepatitis and the genetic characteristics of the individual patient. In another embodiment, the present invention provides a pharmaceutical composition for prevention of hepatocellular carcinoma comprising a therapeutically effective amount of an aromatase inhibitor. Optionally, the pharmaceutical composition may comprise a pharmaceutically acceptable excipient and/or carrier.

A further aspect of the present invention is a method of prophylactic treatment with one or more aromatase inhibitors in a Caucasian male human subject diagnosed as being at risk for liver cancer in order to prevent or delay development of hepatocellular carcinoma comprising administering to a diagnosed subject a pharmaceutical composition comprising (a) a therapeutically effective amount of an aromatase inhibitor, (b) a therapeutically effective amount of an anti-cancer agent, and, optionally, a pharmaceutically acceptable excipient and/or carrier.

In one embodiment, the present invention is directed to methods for the prevention of hepatocellular carcinoma a male Caucasian subject in need thereof. In another embodiment, the present invention is directed to methods for the prevention of hepatocellular carcinoma a male Caucasian subject diagnosed as being in a precancerous condition.

The methods of the present invention are based on the step of selectively inhibiting aromatase (CYP19A1) in the treated subject. According to one embodiment, the inhibition of aromatase (CYP19A1) may be achieved by inhibiting the activity of aromatase using selective aromatase inhibitors that function to irreversibly inhibit aromatase or to reversibly inhibit aromatase by competitive mechanisms. According to one embodiment, the inhibition of aromatase (CYP19A1) may be achieved by inhibiting the expression of the aromatase gene using RNAi or related inhibitory RNAs.

These and other features are explained more fully in the embodiments illustrated below. It should be understood that in general the features of one embodiment also may be used in combination with features of another embodiment and that the embodiments are not intended to limit the scope of the invention.

### BRIEF DESCRIPTION OF THE DRAWINGS

The various exemplary embodiments of the present invention, which will become more apparent as the description proceeds, are described in the following detailed description in conjunction with the accompanying drawings, in which:

FIG. 1 depicts the genetic variation in the expression of the Cdc20 gene product in liver of 42 BXD strains of mice.

FIG. 2 illustrates that the top 13 covariate genes with cdc20 in BXD female liver are all cell cycle-mitosis genes which is highly significant based on the fact that there are <775 cell cycle-mitosis genes of the total ˜24,000 gene genome that yields an expected frequency of one in thirty.

FIG. 3 depicts the cell cycle-mitosis network in the liver of both sexes of BXD recombinant inbred mouse strains. The expression of systems genetics network genes can show an either positive or negative covariance but essentially all of the illustrated network interactions in this and following figures show positive correlation coefficients wherein dark lines indicates a correlation coefficient >0.7, and light line indicates a correlation coefficient >0.5.

FIG. 4 depicts the BXD female mouse spleen cell cycle-mitosis network of genes whose expression is covariant with Cdc20.

FIG. 5 is a chart showing the chromosome 9 eQTL for BXD lung cell cycle-mitosis network of genes that show covariant expression with Cdc20.

FIG. 6 is a chart showing the eQTLs for BXD spleen cell cycle-mitosis network for genes that show Cdc20 expression covariance. The chromosome 15 eQTL has high significance.

FIG. 7A is a chart showing the BHHBF2 adipose tissue cell cycle-mitosis network eQTL with sexual dimorphism in females. FIG. 7B is a chart showing the BHHBF2 adipose tissue cell cycle-mitosis network eQTL with sexual dimorphism in males.

FIG. 8A shows that the BXD female liver cell cycle-mitosis network has a chromosome 2 eQTL. FIG. 8B shows that the cell cycle-mitosis network in BXD male liver has eQTLs that are polygenetic with suggestive eQTLs on chromosomes 4, 6, and 8.

FIG. 9 shows that the breast cancer cell cycle-mitosis network has a significance of 4.23×ê−26 using a search of the NZB×FVB−Nw breast cancer database for the top 500 genes that show expression covariance with Cdc20 as analyzed using GoTree of WebGestalt.

FIG. 10 shows that in the human liver both sex database a total of 47 cell cycle-mitosis network genes with correlation coefficients >0.5 were covariate with Cdc20.

FIG. 11 shows the results of GWAS SNP analysis performed concerning the network of 47 covariate cell cycle-mitosis genes of the Caucasian human liver of both sexes. It shows the most significant GWAS SNPs on chromosomes 9, 15 and 18. The GWAS SNP of chromosome 18 is not associated with a gene whereas the GWAS SNPs of chromosomes 9 and 15 are gene associated.

FIG. 12 shows the results for the Caucasian female dataset and that a chromosome 9 GWAS SNP >8.0−log P for the Astn2 gene is female specific. In addition, the data show that there are multiple additional GWAS SNPs greater that 4.0−log P exist to be considered.

FIG. 13 shows the results for the Caucasian male dataset and that a chromosome 15 GWAS SNP greater than 8.0−log P for the Aro1 gene is male specific. The data also show that multiple additional GWAS SNPs greater that 4.0−log P exist to be considered.

### DETAILED DESCRIPTION

The meanings of the terms used in the specification are as follows:

The term “aromatase” refers to an enzyme of the cytochrome P450 superfamily (CYP19A1), whose function is to aromatize androgens to produce estrogens. Aromatase is predominantly located in the endoplasmic reticulum of the cell and tissue specific promoters that are in turn controlled by hormones, cytokines, and other factors regulate its activity. The principal transformations catalyzed by aromatase are the conversion of androstenedione to estrone and testosterone to estradiol. Aromatase can be found in many tissues including liver, gonads, brain, adipose tissue, placenta, blood vessels, skin, bone and endometrium as well as in tissue of endometriosis, uterine fibroids, and various cancers.

“Aromatase inhibitors” inhibit aromatase (estrogen synthase), a membrane-bound enzyme complex that catalyzes the conversion of androgens to estrogens. Aromatase inhibitors include third-generation aromatase inhibitors, such as anastrozole (Arimidex™) exemestane (Aromasin™), and letrozole (Femara™). These third generation aromatase inhibitors have brought about a major change in the therapeutic approach to patients with estrogen-sensitive cancers, such as breast cancer. Such aromatase inhibitors are very specific in their action. Some inhibitors, such as Exemestane, are irreversible steroidal inhibitors that form a permanent and deactivating bond with the aromatase enzyme whereas others, such as Anastrozole, are non-steroidal inhibitors that decrease estrogen synthesis by reversible competition for the aromatase enzyme

“Candidate gene” is a gene or genetic element that is being tested for an association between the gene and a trait of interest. The candidate gene may be an ortholog of a gene known or suspected to be associated with the trait of interest in a different species. As used herein, the term “associated with” in connection with a relationship between a genetic marker (SNP, haplotype, insertion/deletion, tandem repeat, etc.) and a phenotype refers to a statistically significant dependence of marker frequency with respect to a quantitative scale or qualitative gradation of the phenotype. A marker “positively” correlates with a trait when it is linked to it and when presence of the marker is an indicator that the desired trait or trait form will occur in an organism comprising the marker. A marker negatively correlates with a trait when it is linked to it and when presence of the marker is an indicator that a desired trait or trait form will not occur in an organism comprising the marker. For the purposes of the present invention, the term “marker” refers to any genetic element that is being tested for an association with a trait of interest, and does not necessarily mean that the marker is positively or negatively correlated with the trait of interest. Thus, a marker is associated with a trait of interest when the marker genotypes and trait phenotypes are found together in the progeny of an organism more often than if the marker genotypes and trait phenotypes segregated separately.

“Candidate network” and “candidate systems genetics network” is a set of covariate expressed genes with a common function that are initially identified as a group of genes whose expression is covariant and whose function is shared in common and is selected for testing for an association between candidate genetic regulators and the network.

“Carcinogenesis” or “oncogenesis” or “tumorigenesis” is the multi-stage process by which normal cells are transformed into cancer cells. The key elements of carcinogenesis involve the sequential accumulation of mutations that activate oncogenes and disrupt suppressor genes combined with multiple rounds of clonal selection and clonal evolution. Transient and stable epigenetic events also facilitate the development of cancer. This process can require 10 to 20 years to evolve. The transition from a premalignant stage to a malignant stage in epithelial carcinogenesis is associated with the acquisition of invasiveness and the potential to metastasize.

“Correlation analysis” refers to a correlation-based similarity analysis including a correlation analysis using Pearson's correlation coefficient (PCC) including the related Spearman's rho and Kendall's tau known in the art. “Pearson Correlation Coefficient” or “PCC” refers to the measure of the correlation between two variables and in particular reflects the degree of linear relationship between the two variables.

The term “combination therapy” can mean concurrent or consecutive administration of two or more agents. For example, concurrent administration can mean one dosage form in which the two or more agents are contained whereas consecutive administration can mean separate dosage forms administered to the patient at different times and maybe even by different routes of administration.

“Computer system” refers to the hardware means, software means and data storage means used to compile the data of the present invention. The minimum hardware means of computer-based systems of the invention may comprise a central processing unit (CPU), input means, output means, and data storage means. Desirably, a monitor is provided to visualize structure data. The data storage means may be RAM or other means for accessing computer readable media of the invention

“Effective amount,” “therapeutically effective amount” or “pharmaceutically effective amount” of an agent or compound as provided herein refers to a nontoxic but sufficient amount of the agent or compound to provide the desired therapeutic effect. As will be pointed out below, the exact amount required will vary from subject to subject, depending on age, general condition of the subject, the severity of the condition being treated, and the particular agent or compound administered, and the like. An appropriate “effective amount” in any individual case may be determined by one of ordinary skill in the art by reference to the pertinent texts and literature and/or using routine experimentation.

“eQTL” or eQTG” means a QTL or QTG that signifies the data are derived from gene expression studies using microarray technologies.

“Estrogens” mean a group of estrogenic sex hormones present in both men and women. The three major naturally occurring estrogens are estrone (E1), estradiol (E2), and estriol (E3). All of the different forms of estrogen are synthesized from androgens, specifically testosterone and androstenedione, by the enzyme aromatase.

“Gene chip”, “DNA microarray”, “nucleic acid array”, and “gene array” are used interchangeably herein. Gene chips, or microarrays, are large-scale gene expression monitoring technologies, used to detect differences in mRNA levels of thousands of genes at a time, thus speeding up dramatically genome-level functional studies. Microarrays are used to establish gene expression characteristics of specimens. Microarray data and analysis methods are well known in the art. Variants of DNA microarray technology are also known in the art. For example, cDNA probes of about 500 to about 5,000 bases long can be immobilized to a solid surface such as glass using robot spotting and exposed to a set of targets either separately or in a mixture. Alternatively, an array of oligonucleotides of about 20-mer to about 25-mer or longer oligos or peptide nucleic acid (PNA) probes is synthesized either in situ (on-chip) or by conventional synthesis followed by on-chip immobilization. The array is exposed to labeled sample DNA, hybridized, and the identity and/or abundance of complementary sequences is determined.

“Gene locus” is a location where a gene is coded on a chromosome. Usually, a gene locus is a region on a chromosome to be transcribed to a continuous poly RNA chain by RNA polymerase; however, the term “a gene locus” is sometimes used to include a region regulating transcription. Furthermore, a region consisting of exons, which code a single protein and introns between the exons, is sometimes referred to as a gene locus. At least, any information expressing an existing location of a gene or a marker on a chromosome falls within the gene locus used in the specification.

“Gene network” refers to a network formed by a group of genes whose expression is covariant and whose function is shared in common. The genes of the network interact with each other indirectly (through their RNA and protein expression products) and with other substances in the cell, thereby governing the rates at which genes in the network are transcribed into mRNA.

“GeneNetwork” and “genenetwork.org” refers to a computer database and open source bioinformatics software resource for systems genetics. Data sets in GeneNetwork are typically made up of large collections of genotypes (e.g., SNPs) and phenotypes that are obtained from groups of related individuals, including human families, experimental crosses of strains of mice and rats, and organisms such as Drosophila melanogaster, Arabidopsis thaliana, and barley.

“Gene(s) of interest” means one or more known genes that may be used as a quantitative trait that is being characterized using the method of the present invention. The level of expression of the gene of interest may be determined using any methods known in the art, for example, Northern analysis, RNase protection, array analysis, PCR and the like. The gene of interest or the level of its transcripts is a quantitative trait that is used for further identification of genes that have covariate expression with the gene of interest and a common function that can comprise a network and one or more eQTLs associated with the expression of the network associated with the gene of interest. One or more genes of interest may be used within the method of the present invention. The primary gene of interest used to identify the cell cycle-mitosis network in the current invention is Cdc20. Additional genes of interest for the cell cycle-mitosis network can be selected genes that comprise the network.

“Genome” refers to all the genetic material in the chromosomes of a particular organism. Its size is generally given as its total number of base pairs. Within the genome, the term “gene” refers to an ordered sequence of nucleotides located in a particular position on a particular chromosome that encodes a specific functional product (e.g., a protein or RNA molecule). In general, an animal's genetic characteristics, as defined by the nucleotide sequence of its genome, are known as its “genotype,” while the animal's physical traits are described as its “phenotype.”

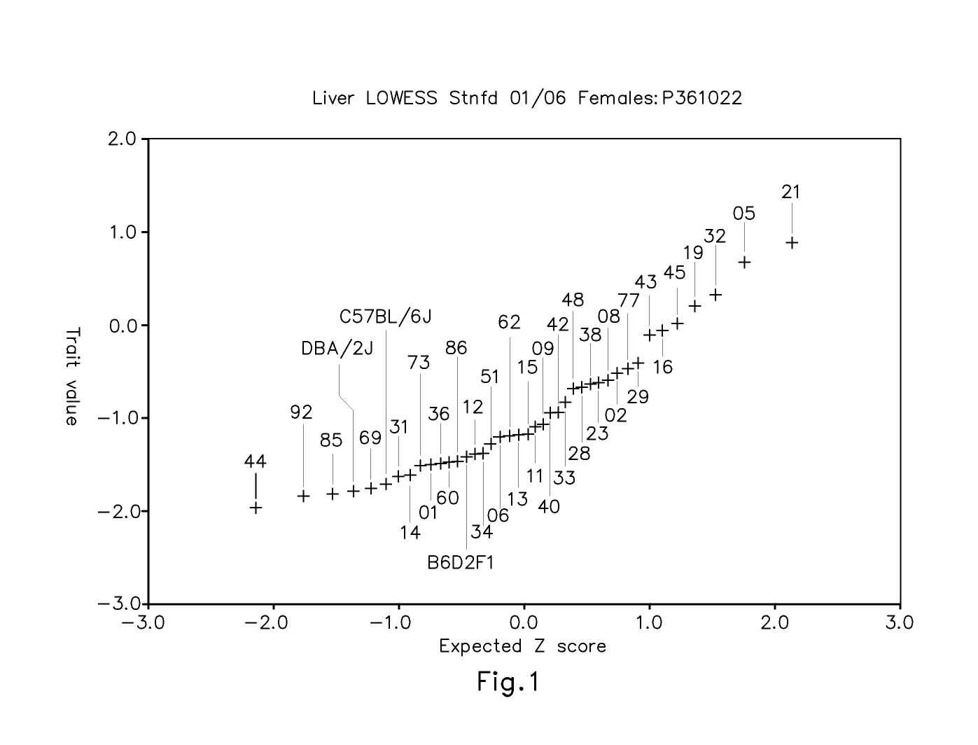

“Genomic coordinate” is one dimensional coordinate used to express relative positions between gene loci on a chromosome, expressing the positions in a direction from 5′ terminal to 3′ terminal (or in a direction from 3′ terminal to 5′ terminal) in one of the chains of a double-stranded DNA constituting a chromosome. As shown in FIG. 1, locations of gene loci are sometimes expressed by corresponding one chromosome to one genomic coordinate.

“Genome-wide association study (or “GWAS”), also known as “whole genome association study” (or “WGAS”), is an examination of many common genetic variants in different individuals to see if any variant is associated with a trait. GWAS typically focus on associations between single-nucleotide polymorphisms (SNPs) and traits like major diseases. These studies normally compare the DNA of two groups of participants: people with the disease (cases) and similar people without (controls). Each person gives a sample of DNA, from which millions of genetic variants are read using SNP arrays. If one type of the variant (one allele) is more frequent in people with the disease, the SNP is then the to be “associated” with the disease. The associated SNPs are then considered to mark a region of the human genome, which influences the risk of disease. In contrast to methods, which specifically test one or a few genetic regions, the GWA studies investigates the entire genome. The approach is therefore non-candidate-driven in contrast to gene-specific candidate-driven studies. GWA studies identify SNPs and other variants in DNA that are associated with a disease, but cannot on their own specify which genes are causal.

“Hepatocellular carcinoma” refers to a type of liver cancer that is a primary malignancy of the hepatocyte, generally leading to death within 6-20 months. Hepatocellular carcinoma (HCC) most frequently arises in the setting of chromic viral hepatitis and cirrhosis, appearing 20-30 years following the initial insult to the liver. Chronic alcohol consumption and cirrhosis also are cofactors that increase the development of HCC in patients with chronic viral infection. The extent of hepatic dysfunction limits treatment options and prognosis of HCC patients is very poor with most studies reporting a five year survival rate of from ˜5% to <20% depending on the characteristics of the viral hepatitis and the genetics of the individual patient.

“Likelihood ratio statistic” or “LRS” means a measurement of the association or linkage between differences phenotypes and differences in particular DNA sequence (marker sequence). These values are used in genetic maps of traits, usually plotted on the y-axis. Values above 10 to 15 will usually be worth attention for simple interval maps. The term “likelihood ratio” is used to describe the relative probability of two different explanations for variation in a trait. The first explanation (or model or hypothesis Hi) is that the differences in the trait ARE associated with that particular DNA sequence difference. The second “null” hypothesis (Hₙᵤₗₗ or H₀) is that differences in the trait are not associated with that particular DNA sequence. We can compute the probability of these two different explanations and use this ratio as our score. If model A is 1000 times more probable than model B, then the ratio of the odds are 1000:1 and the logarithm of the odds ratio is 3.

“Linked Function Network Regulator” or “LFNR” concerns the principle that provides a unique approach to define the best set of candidate genetic regulators for a network of interest. LFNRs are identified by screening a plurality of eQTLs for the network of interest of multiple populations of various species, sexes, tissues, cells, and experimental situations and identifying a linked function shared by the candidate eQTGs associated with the network of interest in the populations. The term “linked function” has a broader applicability than the term “common function” that is used relative to network characteristics. Whereas the term common function is used to define genes with shared gene ontology; the term-linked function includes both genes that share a common function and genes that have the potential to impact or influence the common function. An example of such a distinction is evident concerning the cell cycle-mitosis network. Specifically, genes that share a common function with the cell cycle-mitosis network gene of interest—Cdc20, also have a direct role in the mechanisms of the cell cycle whereas Linked Function Network Regulators for the cell cycle-mitosis network can include genes such as Aro1 that regulate the synthesis of estrogen that can influence the expression and/or activity of multiple cell cycle genes. LFNRs can include eQTGs identified in studies using genetic variation panels of interbreeding animals or animal sets and GWAS SNPs identified in studies of human populations.

“Locus” or “loci” refers to the site of a gene on a chromosome. Pairs of genes, known as “alleles” control the hereditary trait produced by a gene locus. Each animal's particular combination of alleles is referred to as its “genotype”.

“LRS significant threshold” means the approximate LRS value that corresponds to a genome-wide p-value of 0.05, or a 5% probability of falsely rejecting the null hypothesis that there is no linkage anywhere in the genome. This threshold is computed by evaluating the distribution of highest LRS scores generated by a set of 2000 random permutations of strain means. For example, a random permutation of the correctly ordered data may give a peak LRS score of 10 somewhere across the genome. The set of 1000 or more of these highest LRS scores is then compared to the actual LRS obtained for the correctly ordered (real) data at any location in the genome. If fewer than 50 (5%) of the 1000 permutations have peak LRS scores anywhere in the genome that exceed that obtained at a particular locus using the correctly ordered data, then one can usually claim that a QTL has been defined at a genome-wide p-value of 0.05. The threshold will vary slightly each time it is recomputed due to the random generation of the permutations.

“LRS Suggestive threshold” means the suggestive threshold represents the approximate LRS value that corresponds to a genome-wide p-value of 0.63, or a 63% probability of falsely rejecting the null hypothesis that there is no linkage anywhere in the genome. This is not a typographical error. The Suggestive LRS threshold is defined as that which yields, on average, one false positive per genome scan. That is, roughly one-third of scans at this threshold will yield no false positive, one-third will yield one false positive, and one-third will yield two or more false positives. This is a very permissive threshold, but it is useful because it calls attention to loci that may be worth follow-up. Regions of the genome in which the LRS exceeds the suggestive threshold are often worth tracking and screening. They are particularly useful in combined multi-cross meta-analysis of traits. If two crosses pick up the same suggestive locus, then that locus may be significant when the joint probability is computed. The suggestive threshold may vary slightly each time it is recomputed due to the random generation of permutations.

The term “pathway” refers to a sequence of gene products (proteins) that function in sequence either as individual entities or as part of a complex to mediate a biological function. Typical pathways include metabolic pathways and signaling pathways among many others [See http://en.wikipedia.org/wiki/WikiPathways]. A pathway is distinct from a systems genetics network as used in the current invention.

“Phenotypic trait” refers to the appearance or other characteristic of an organism, e.g., a plant or animal, resulting from the interaction of its genome with the environment. The term “phenotype” refers to any visible, detectable or otherwise measurable property of an organism. The term “genotype” refers to the genetic constitution of an organism. This may be considered in total, or with respect to the alleles of a single gene, i.e., at a given genetic locus. In some embodiments, the markers are candidate genes or genetic elements directly attributable to the phenotypic trait.

A “precancerous condition” or “premalignant condition” is a state associated with a significantly increased risk of cancer resulting from the initiation and progression of the process of carcinogenesis to a certain stage.

“Probe” is a nucleic acid sequence, optionally tethered, affixed, or bound to a solid surface such as a microarray or chip. Probes are generally oligonucleotides of variable length, used in the detection of identical, similar, or complementary nucleic acid sequences by hybridization. An oligonucleotide sequence used as a detection probe may be labeled with a detectable moiety.

“Quantitative trait genes” or “QTGs” means the gene(s) associated with a quantitative trait locus or QTL and underlying trait variation that has the potential to regulate the characteristics of that trait.

“Quantitative trait locus” or “QTL” means a region of any genome that is responsible for some percentage of the variation in the quantitative trait of interest. Within these regions are located one or more genes coding for factors that have a significant effect on the phenotype of the organism. A QTL is generally a stretch of DNA containing or linked to the genes that underlie a quantitative trait. Mapping regions of the genome that contain genes involved in specifying a quantitative trait is done using molecular tags such as Amplified fragment length polymorphisms or single nucleotide polymorphisms (SNPs). This is an early step in identifying and sequencing the actual genes underlying trait variation

“Recombinant inbred strains” have chromosomes incorporate a fixed and permanent set of recombinations of chromosomes originally descended from two or more parental strains. Sets of RI strains are often used to map the chromosomal positions of polymorphic loci that control variance in phenotypes. Chromosomes of RI strains typically consist of alternating haplotypes of highly variable length that are inherited intact from the parental strains. In the case of a typical rodent RI strain made by crossing maternal strain C with paternal strain B, a chromosome will typically incorporate 3 to 5 alternating haplotype blocks with a structure such as BBBBBCCCCBBBCCCCCCCC, where each letter represents a genotype, series of similar genotype represent haplotypes, and where a transition between haplotypes represents a recombination. Both pairs of each chromosome will have the same alternating pattern, and all markers will be homozygous. Each of the different chromosomes will have a different pattern of haplotypes and recombinations. The only exception is that the Y chromosome and the mitochondrial genome, both of which are inherited intact from the paternal and maternal strain, respectively. For an RI strain to be useful for mapping purposes, the approximate position of recombinations along each chromosome need to be well defined either in terms of centimorgan or DNA base pair position. The precision with which these recombinations are mapped is a function of the number and position of the genotypes used to type the chromosomes. RI strains are almost always studied in sets or panels. All else being equal, the larger the set of RI strains, the greater the power and resolution with which phenotypes can be mapped to chromosomal locations. Between 2005 and 2007, virtually all extant mouse and rat RI strains were re-genotyped at many thousands of SNP markers, providing highly accurate maps of recombinations.

“Record” is a unit for handling data stored in a database. As a record, a file in a file system, a record in a relational database, an object in an object-oriented database and the like are suitably used. Using a computer may sometimes refer to data treatable as a single object by using a computer as a record in the specification.

“Remote computer” means a computer, which communicates with a local computer in this system, and is composed of one or more computers. A remote computer may be located at one site, or may be located at two or more sites.

“Single nucleotide polymorphism” or “SNP” refers to a variation in the nucleotide sequence of a polynucleotide that differs from another polynucleotide by a single nucleotide difference. For example, without limitation, exchanging one A for one C, G or T in the entire sequence of polynucleotide constitutes a SNP. It is possible to have more than one SNP in a particular polynucleotide. For example, at one position in a polynucleotide, a C may be exchanged for a T, at another position a G may be exchanged for an A and so on. When referring to SNPs, the polynucleotide is most often DNA.

By the term “siRNA or RNAi” is meant a double stranded RNA molecule which prevents translation of a target mRNA. Standard techniques of introducing siRNA into the cell are used, including those in which DNA is a template from which RNA is transcribed. The siRNA includes a sense nucleic acid sequence, an anti-sense nucleic acid sequence or both. The siRNA is constructed such that a single transcript has both the sense and complementary antisense sequences from the target gene, e.g., a hairpin.

As used herein the term “system” refers to a collection of parts having functional association, for example, an existence separated and extracted from the circumstances as a target of analysis and discussion. Systems include, but are not limited to: for example, scientific systems (for example, physical systems, chemical systems, biological systems (for example, cells, tissues, organs, organisms and the like), geophysical systems, astronomical systems, and the like), social scientific systems (for example, company organization and the like), human scientific systems (for example, history, geography and the like), economic systems (for example, stock price, exchange and the like), machinery systems (for example, computers, apparatus and the like) and the like.

“Systems genetics” or “network genetics” means an emerging new branch of genetics that aims to understand complex causal networks of interactions at multiple levels of biological organization. To put this in a simple context: Mendelian genetics can be defined as the search for linkage between a single trait and a single gene variant (1 to 1); complex trait analysis can be defined as the search for linkage between a single trait and a set of gene variants (QTLs, QTGs, and QTNs) and environmental cofactors (1 to many); and systems genetics can be defined as the search for linkages among networks of traits and networks of gene and environmental variants (many to many). While a gene pathway is a series of genes that work together in series, a systems network is a series of genes where a substantial number of genes interact with each other substantially at the same time. A hallmark of systems genetics is the simultaneous consideration of groups (systems) of phenotypes from the primary level of molecular and cellular interactions that ultimately modulate global phenotypes such as blood pressure, behavior, or disease resistance Changes in environment are also often important determinants of multiscalar phenotypes; reversing the standard notion of causality as flowing inexorably upward from the genome. Scientists who use a systems genetics approach often have a broad interest in modules of linked phenotypes. Causality in these complex dynamic systems is often contingent on environmental or temporal context, and often will involve feedback modulation. A systems genetics approach can be unusually powerful, but does require the use of large numbers of observations (large sample size), and more advanced statistical and computational models. Complex trait analysis and QTL mapping are both part of systems genetics in which causality is inferred using conventional genetic linkage. One can often assert with confidence that a particular module of phenotypes (component of the variance and covariance) is modulated by sequence variants at a common locus. This provides a causal constraint that can be extremely helpful in more accurately modeling network architecture.

“Traits”, “quality traits” or “physical characteristics” or “phenotypes” refer to advantageous properties of the animal resulting from genetics. The terms may be used interchangeably.

“Winsorization” is a statistical procedure that involves the transformation of a dataset by limiting extreme values to reduce the effect of possibly spurious outliers.

### DESCRIPTION OF SPECIFIC EMBODIMENTS

It has been established by recent publications [Begley C G and Ellis L M, Nature 483: 531, 2012; Prinz F, Schlange T and Asadullah K, Nature Drug Discov. 10: 712. 2011] that only ˜25% of preclinical biological research studies published by academics can be reproduced by pharmaceutical companies as part of drug develop endeavors and that in the field of cancer research only 11% reproducibility can be achieved. Preclinical studies relating to systems genetics that rely on the evaluation of large sample populations and complex technologies, such as microarray analysis, risk similar or greater problems of reproducibility. Therefore new data validation methods, processes and platforms need to be used to assure that preclinical systems genetics outcomes are valid and that they translate to human application for drug discovery.

The present innovation relates generally to methods, processes and platforms for use to validate systems genetics networks of genes that share a common function and to define their genetic network regulators for translation to humans as disease-specific drug targets. In particular, this invention relates to methods, procedures and platforms for using both microarray-based gene expression data and bioinformatics analysis to identify gene-gene interactions, gene-phenotype interactions, and linked-function network regulators of complex traits in large populations that show genetic variation.

To discover systems genetics networks, investigators typically select a gene of interest, such as Cdc20—a mitotic spindle checkpoint gene, or one of a set of genes that are components that have a known biological function, such as Cdc20, Aurka, Prc1, Birc5, Plk4, Plk1, Ccnb1, Cdca1 and Ncaph—cell cycle-mitosis genes. A gene expression database is then developed using microarray technologies to define gene expression covariates with the gene of interest for a specific genetic variation panel of cells, tissues, or animals, such as BXD recombinant inbred mice. Gene ontology analysis systems can then be used to define expression covariate sets that share functions in common in such a population. Manual and/or computer-based approaches are typically used to accomplish such bioinformatics procedures to identify systems genetic networks. Similar approaches can be used to identify systems genetics networks of phenotypes. Once a specific systems genetics network is identified, searches for the eQTL and eQTGs that have the potential to serve as regulators of the networks are then typically undertaken.

More specifically, screening for a single gene or group of genes of interest typically employs bioinformatics to analyze gene expression or other types of databases made up of genetically diverse collections of specimens from large populations of genotypes. The databases commonly used include GeneNetwork, BisoGenet, Cytoscape, VisANT, Osprey and Biological Networks, which are generally able to build and visualize biological network representation of relationships among biomolecules. Data repositories such as NCBI's Entrez Gene and Ensembl maintain annotation on whole genomes, including sequences, gene location, transcripts, classification and links to several external databases. Data retrieved from high-throughput experiments and literature are available from several databases, such as, DIP, BIND, HPRD, BioGRID, MINT and Intact, which represent the major repositories of protein-protein interactions from multiple organisms. Databases like KEGG, Reactome, BioCyc, NCI Nature PID and others provide information on both metabolic and signaling pathways. Such databases are used to screen for genes within a microarray expression dataset that co-vary with the gene of interest and preferably with other related transcripts for that gene. Then, all the covariantly expressed genes with correlation coefficients greater than or equal to 0.5 can be exported to a gene ontology analysis system such as WebGestalt [a “WEB-based GEne SeT AnaLysis Toolkit” at http://bioinfo.vanderbiltedu/webgestalt/]. Using such a geneontology analysis approach, it is possible to determine which if any of the covariantly expressed genes share a common function and thereby have the characteristics of a systems genetic network. If such analyses define a functionally linked set of genes that show good co-variance, it can be defined as a candidate systems genetics network in that particular dataset. Rarely are multiple varieties of databases used in such studies. Once such data are developed using animal systems, their applicability to humans is typically sought by use of GWAS SNP analysis and related studies.

This invention therefore relates to new methods, processes and platforms to be used to assure that preclinical systems genetics information concerning biological networks are valid and to define their genetic network regulators for translation to humans as disease-specific drug targets.

### Section 1. The Multiple Criteria Validation (MCV) Process for Systems Genetics Networks Providing a Foundation of the Definition of Linked-Function Network Regulators (LFNRs).

In the first embodiment, once a candidate systems genetics network of genes that share a common function has been defined, and needs to be validated, the following process is to be used. The steps of the MCV process can be accomplished using GeneNetwork or any other substantially similar bioinformatics tool that can perform related functions.

In one embodiment, the MCV process is used to validate candidate systems genetics networks that function in multiple cell types, in multiple tissues, in multiple species and in both sexes. In another embodiment, the process is used in situations where only one tissue or cell type expresses the candidate network such that the requirements of the MCV process are met except for the step requiring two or more tissue or cell types.

The MCV method comprises the following steps:

1. Determining that a specific candidate systems genetics network of covariate expressed genes that share a common function, exists in two or more tissue or cell types;
2. Determining that the specific candidate network exists in two or more databases developed by different laboratories and/or investigators;
3. Determining that the specific candidate network can be replicated in databases developed using two or more different microarray technologies and/or platforms;
4. Determining that the specific candidate network exists in databases developed using two or more different animal species and/or strains;
5. Determining that the specific candidate network can be reproduced in databases developed using at least two or more different microarray data normalization systems;
6. Determining that the specific candidate network has one or more suggestive or significant eQTLs; and
7. Determining that the specific candidate network exists substantially more in tissues and/or cells that are physiologically relevant than in tissues and cells that are not physiologically correct (as a negative control).

In one embodiment, one or more of steps 1 through 7 are accomplished using GeneNetwork. It is not necessary that the candidate network be proven to exist in every possible example because some databases may have intrinsic problems that might abrogate the analysis and in some examples the network may actually not exist because of the biological characteristics of the specimen examine

In another embodiment, the method used as part of the MCV process to validate the significance of the defined network comprises the following steps:

1. Determining that the specific candidate network of covariate expressed genes that share a common function have correlation coefficients greater than or equal to 0.5, 0.6, 0.7. 0.8, 0.9 or higher exists in two or more tissues or cell types. In one embodiment, the two or more tissues or cell types is determined using the mouse BXD genetic reference population and then other related animal populations;
2. Determining that the specific candidate network exists in two or more databases developed by different laboratories and/or investigators;
3. Determining that the specific candidate network can be replicated in databases developed using two or more different microarray technologies and/or platforms and optimally that more than one transcript for the gene of interest be used to identify and define specific candidate networks in each database;
4. Determining that the specific candidate network can be reproduced in databases developed using at least two different microarray data normalization systems, such as, MASS and RMA;
5. Determining that the specific candidate network exists in databases developed using two or more different animal species and/or strains, i.e., BXD mouse strains or various F2 mouse populations, or different animal species, such as, rats;
6. Determining that the specific candidate network shows one or more suggestive or significant eQTLs at least in the most significant examples of all the above situations; and
7. Determining that the specific network exists substantially only in tissues and/or cells that are physiologically relevant and not in tissues and cells that are not physiologically correct (as a negative control).

In one embodiment, one or more of steps 1 through 7, the specific candidate network of covariate expressed genes with a shared common function are selected as those with correlation coefficients greater than or equal to 0.7 in two or more tissues or cell types. In another embodiment, the specific candidate network of co-variant expressed genes are selected as those with correlation coefficients greater than or equal to 0.9 in two or more tissues or cell types.

In another embodiment, one or more of steps 1 through 7, the gene components of a specific candidate network can vary in each situation while in all situations being part of a common function of the network. In the cell cycle-mitosis network example, of the ˜775 cell cycle-mitosis genes known to exist, the network in each situation typically contains 30 to 60 cell cycle-mitosis genes of which ˜25 to 50% are typically shared in common with other the network in other situations and the other percentages are distinct for that situation.

In one embodiment, one or more of steps 1 through 7 are accomplished using a computer bioinformatics system. In any of steps 1 through 7, it is not necessary that the candidate network be proven to exist in every possible example because some databases may have intrinsic problems that might abrogate the analysis and in some examples the network may actually not exist because of the biological characteristics of the specimen examined.

Once all the above requirements are substantially completed as part of the MCV process, the candidate network can be deemed to be validated with a defined degree of certainty and the network in its characteristics in all the different parameters used for its validation can then to be used as the foundation to evaluate and test the LFNR principle using the LFNR platform, as described in Section 2 below.

### Section 2. Linked-Function Network Regulators (LFNRs)

Once a candidate systems biology network has been validated successfully using the MCV process (Section 1), a vast amount of information about the network will be available to serve as the foundation for studies to define the genetic regulator(s) of the network. This Section 2 describes the LFNR principle and the LFNR platform to be used to establish the genetic mechanism(s) that serves to regulate the characteristics of the validated systems genetics network.

In a further embodiment, the LFNR principle and LFNR platform are used as part of a method to determine which candidate eQTGs in non-human animal populations (and subsequently candidate GWAS SNPs in human populations) have the highest potential to regulate the systems genetics network of interest. This method is based on the following:

1) eQTLs derived from analysis of multiple representative specimen databases defined as part of the MCV process are analyzed in detail using bioinformatics tools (such as, www.genenetwork.org) to screen for all the candidate eQTGs associated with the eQTLs for the network in each situation;
2) Since networks validated using the MCV process, such as the cell cycle-mitosis network (see Section 3 that follows), show species, strain, sex, and tissue specificity, the eQTLs and candidate eQTGs for these networks will also show species, strain, sex and tissue specificity. This means that there can be complexity in the number of candidate eQTGs that have the potential to regulate such a network; and
3) To resolve such complexity, the LFNR principle and the LFNR platform have been developed as key parts of this invention:

The LFNR principle of the present invention first states that a single or a small subset of candidate eQTGs for a systems genetics network, which has been characterized in multiple situations per the MCV process described herein, will be found to share a linked function.

The LFNR principle further states that those candidate eQTGs that share that linked function represent the most probable regulatory eQTGs or linked-function network regulators (LFNRs) in their respective situations. Such eQTLs may act in cis (locally) or trans (at a distance) to a gene.

Once candidate eQTGs associated with eQTLs for a specific network are identified in multiple situations and compiled, all cis candidate eQTGs (and trans candidate eQTGs in some situations) are compiled and analyzed for each situation to identify a linked function shared by selected candidate eQTGs in each situation.

Once a linked function is identified, the complexity of defining the regulatory eQTGs for such a network in multiple tissues is markedly simplified to a single or a small subset of eQTGs defined to represent the most probable network regulators, i.e., LFNRs.

In the present invention, use of the LFNR principle relative to the cell cycle-mitosis network has established that a small subset of the candidate cis eQTGs have a linked function and that linked function is actually shared with the function of the network regulated by the LFNRs. Therefore, the LFNRs for the cell cycle-mitosis network are cell cycle or mitosis gene products.

A key insight regarding such LFNRs is that in every situation (such as when using different tissue or cell preparations) that expresses a specific network such as the cell cycle-mitosis network, it is possible to have a distinct LFNR associated with the network. Therefore, in an analysis of a specific network in multiple tissues as prescribed with the MCV process, a variety of different LFNRs for a given network can be found to exist so long as they all have a shared linked function.

One embodiment of the LFNR principle is the LFNR platform of the present invention. The LFNR platform represents the methods and systems as described herein to be used to implement the LFNR principle.

In another embodiment concerning the LFNR platform, using tissue specimens derived from recombinant inbred BXD mice, an eQTL for a specific systems genetics network typically encompasses approximately 30 megabases of DNA and is associated with an average of approximately 150 genes that represent candidate eQTGs. Such candidate eQTGs can have either trans or cis characteristics that commonly are present with a relative ratio of 10:1. In this regard, published evidence suggests that those candidate eQTGs with cis characteristics have preferential functional significance. [See, e.g., Doss S, Schadt E E, Drake T A, Lusis A J. Cis-acting expression quantitative trait loci in mice. Genome Res 15:681-91, (2005)].

In another embodiment concerning the LFNR platform, once the LFNRs for a specific network has been defined from a large set of animal specimens using the MCV process, it is possible to use that information concerning LFNR characteristics of a specific network for translation to human specimens and datasets in Section 5 infra.

In another embodiment based on the composite of information derived from non-human animal studies described above, the method for translation to humans comprises the steps:

1. Establish that the specific network of interest exist in human populations of one or more type of cell and/or tissue;

2. Perform GWAS SNP analysis for the specific network in those human specimens using GeneNetwork or comparable bioinformatics analysis tools to identify if any suggestive or significant GWAS SNP set is enriched in genes that share the linked function observed in the animal LFNRs for that same network;

3. Define the GWAS SNPs that represent the best candidate LFNRs for the specific network in a population of one or more human tissues or cell preparations. As part of this process an in depth analysis of the known function of all the candidate LFNRs is performed using online references sites, such as PubMed, to assure that all linked-functions among the candidate LFNRs are identified; and

4. Determine which candidate GWAS SNP LFNRs are of highest statistical and functional significance and thereby represent the most probable network regulators for the network of interest.

In one embodiment, for human GWAS SNPs, a statistic greater than 4.0−log P is considered to be of possible significance. In another embodiment, for human GWAS SNPs, a statistic greater than 5.0−log P is considered to be of probable significance. In another embodiment, for human GWAS SNPs, a statistic greater than 8.0−log P is considered to be significant.

In one embodiment, the individuals are human subjects. In another embodiment, the human database will provide such information including GWAS SNP data for all subgroups of a population (e.g., ethnic groups in the human population), where designated subgroups can be based on age, gender, ethnicity, geography, race, or any other identifiable population group or subgroup.

The LFNR principle and the LFNR platform defines functionally important systems genetics network regulators that can server as targets for drugs with the ability to modulated network characteristics and thereby biological functions that have human disease relevance such as in cancer prevention and cancer therapy.

One embodiment of the invention is directed to accessing one or more human sets of data representing gene expression data. In one embodiment, each data set is a compilation of data obtained from at least 100, 200, 300, 400, 500, 1,000, 2,000, 3,000, 4,000, or greater than 5,000 subjects. The database/data sets used to screen for GWAS SNPS of gene networks within a microarray expression dataset that covary with the network of interest is a compilation of expression data obtained from whole genome expression arrays of from specially designed expression arrays of 100, 200, 300, 400, 500 or >1000 genes such as an expression array for human cancer genes.

In other embodiments, the GWAS SNP outcomes are accessed using a computer system designed to implement bioinformatics tools.

### Section 3. The Cell Cycle-Mitosis Network and its Genetic Regulators: Proof of Principle for the MCV Process and the LFNR Platform.

Using the MCV process combined with the LFNR platform and meeting all its associated steps listed in the process of Sections 1 and 2, a cell cycle-mitosis network and its LFNRs has been identified and characterized to validate these processes and platforms.

The discovery of the cell cycle-mitosis in many animal specimens with species, strain, sex and tissue specificity and associated LFNRs serves to validate the MCV process and the LFNR platform and thereby serves as proof of principle for this invention.

The present invention provides for methods to identify and validate the cell cycle-mitosis network and associated LFNRs in different non-human animals (and subsequently in humans—see Section 5 that follows).

The cell cycle-mitosis network exists in all studied proliferative tissues and cells and is extremely robust being evident in databases developed by many laboratories and using multiple microarray platforms and normalization systems. Each tissue, cell system, sex and species/strain shows an impressive cell cycle-mitosis network. The cell cycle-mitosis network shows definitive evidence of genetic regulation since in searches of >500 genes as potential network keys, the inventors have found no other network of comparable significance.

The average total number of cell cycle genes in humans, mice and rats is approximately 775, including approximately 210 mitosis genes (see amigo.geneontology.org). The cell cycle-mitosis network has been shown to exist in more than 10 animal tissues, including liver, lung, spleen, kidney hematopoietic stem cells, thymus, cartilage, the eye, adipose tissue and lymphocytes. The network was first discovered by the detection of genes with a common function whose expression is covariant with Cdc20. While other genes that are part of the cell cycle-mitosis network in specific tissues can also be used as the key or gene of interest to identify the network; they typically have shown moderately less robust results.

The cell cycle-mitosis network of the present invention was initially discovered using the UNC Agilent G4121A Liver Lowess Stanford databases in GeneNetwork website (http://www.genenetwork.org/webqtl/main.py). The data set of GeneNetwork was searched for genes that show expression covariance with Cdc20, a key mitotic spindle checkpoint gene, with a correlation coefficient of greater than 0.5 (FIG. 1). Thereafter, multiple additional databases were employed as required by the MCV process and as explained in the following compilation of embodiments.

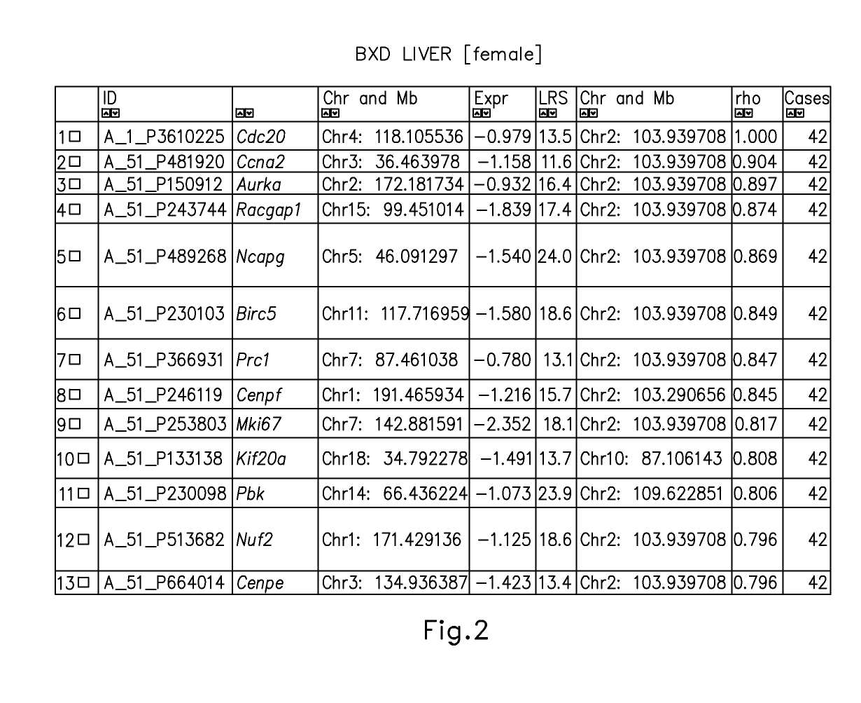

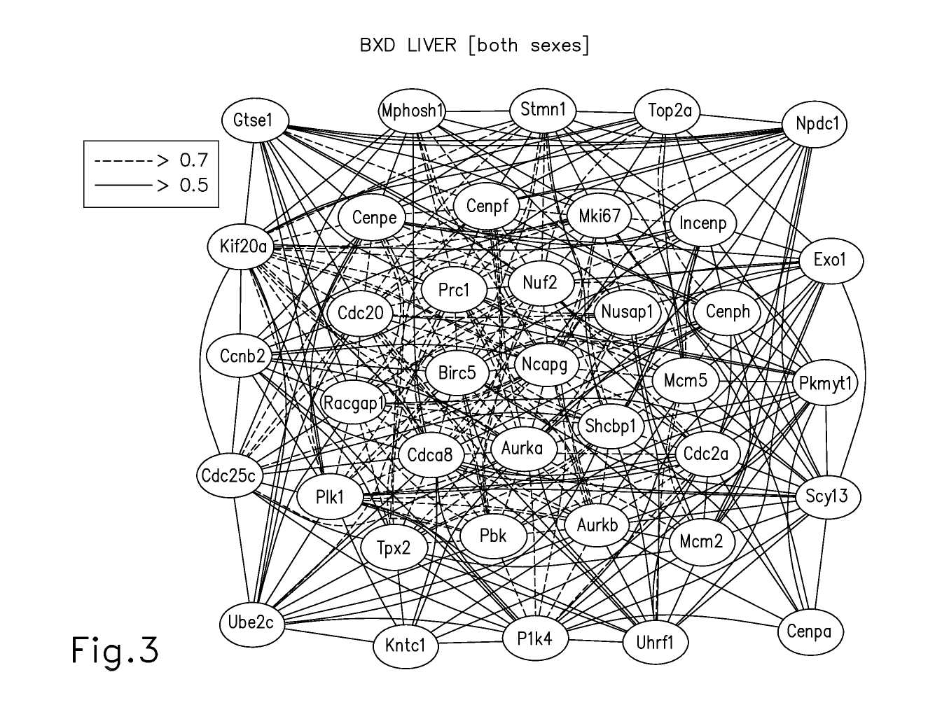

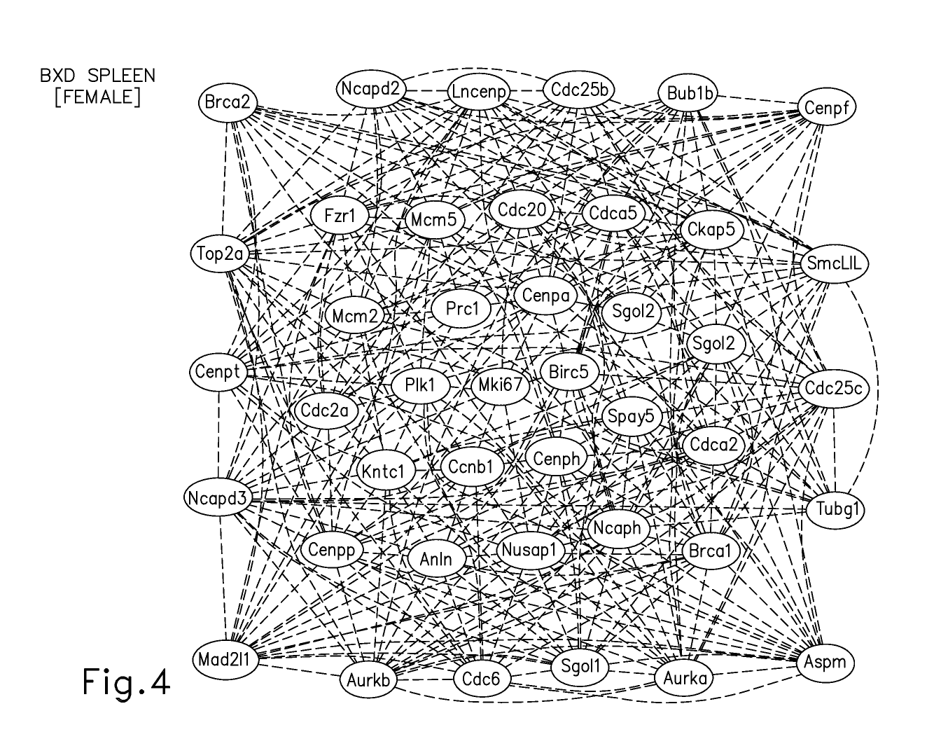

As an example of the high level of Cdc20 expression covariance that is evident, the inventors found that the top 13 genes whose expression is covariant with Cdc20 in livers of female BXD strains of mice are all linked to the cell cycle and/or mitosis and that a total of 48 cell cycle-mitosis genes are covariate with Cdc20 with a p=1.99ê−23 (FIG. 2 and Table 1). FIG. 3 illustrates the characteristics of interaction of all the cell cycle-mitosis network genes for this dataset by use of the network graph function of GeneNetwork (genenetwork.org).

The overall significance of the cell cycle-mitosis network gene covariance in tissues and cells is documented in Table 1 for numerous tissues in mice and rats.

TABLE 1
Cell Cycle - Mitosis Network Significance in
Selected Tissues and Cells.

| Tissues | Total # of genes | Highest significance* |
|---|---|---|
| BXD Mouse Liver - Female | 48 | p = 1.99e\hat{ }−23 |
| BFHBF2 Mouse Liver - | 43 | p = 1.56e\hat{ }−9 |
| Female | | |
| BXD Mouse Lung | 76 | p = 3.34e\hat{ }−29 |
| BXD (IoP) Mouse Spleen | 42 | p = 3.09e\hat{ }−26 |
| BXD Mouse Eye | 44 | p = 6.78e\hat{ }−8 |
| BHHBF2 Mouse Adipose | | |
| Tissue | | |
| Female | 53 | p = 2.01e\hat{ }−29 |
| Male | 46 | p = 4.53e\hat{ }−21 |
| HXBBXH Rat Liver | 42 | p = 5.10e\hat{ }−13 |

*Established using the Vanderbilt WebGestalt GoTree analysis system.

FIG. 4 illustrates another example of the cell cycle-mitosis network. The figure shows the genes and their interconnections in the spleen of BXD mice. One important aspect of this set of discoveries is that the cell cycle-mitosis that exists in proliferative tissues is distinct in each situation. More specifically, the genes that comprise the cell cycle-mitosis network include different combinations of genes in each situation so that there is species, strain, sex and tissue specificity. In this regard, the composition of genes of cell cycle-mitosis network in the spleen is distinct from that in the liver, and so forth.

Concerning the cell cycle-mitosis network observed in many different situations relative to strain, sex, and tissue, the composition of cell cycle-mitosis genes consist of two subsets wherein one subset exists in which many network genes are shared in different situations whereas in the other subset other network genes tend to be distinct in each situation as described in the following paragraphs.

The 36 most common cell cycle-mitosis network members that are evident as complied using almost all studied specimens include: Cdc20, Aurka, Nuf2, Cenpf, Nek2, Nusap1, Tpx2, Ube2c, Ccna2, Cenpe, Cdca8, Prc1, Mki67, Ccnb2, Aurkb, Spag5, Birc5, Cenph, Racgap1, Sgol1, Kif20a, Cdca5, Kntc1, Plk4, Cenpa, Plk1, Cdc2a, Ncapg, Incenp, Top2a, Npdc1, Ncaph, Ktcn2, Cdca3, Cdca1 and Ccnb1

Another 130 cell cycle-mitosis network members that are less frequently evident in various tissues include: Cdc2, Cdc25c, Mphosh1, Uhrf1, Scyl3, Pbk, Shcbp1, Pkmyt1, Exo1, Gtsel, Stmn1, Chek, Cdc451, Cenpt, Mad2l1, Zwilch, Smc2, Anin, Cdc42, Ncapd2, Bub1b, Ttk, Anapc5, Cdca4, Aspm, Kif22, Cdc1, Ckap21, Zwint, Wee1, Cdk2, Pstpip1, Cdt1, Fbxo5, Sertad2, Dbf4, Lig1, Smc2l1, Spag1, Cenpp, Solt, Fshprh1, Ccnf, Cks2, Brrn1, Cdc91l1, Ereg, Cks1b, Pardbg, Psen, Htatip2, Katna1, Rbbp8, Spin, Camk2d, Tgfb2, Pola, Nfatc1, Trp53bp1, Tubb5, Ndc1, Ncapd3, Spc24, Numa1, Cenpb, Cenpm, Smc4, Cenpi, Smc2, Cep55, Tipin, Ndc80, Kifc1, Cdc123, Cdca2, Spc25, Kif23, Ccna2, Stmn1, Dlgap5, Kif4a, Timeless, Aurkc, Cdc25a, Cdc6, Espl1, Kif2c, Cenpn, Cdca3, Brac2, Fzr1, Tubg1, Ckap5, Numa1, Nudc, Scyl3, Tacc3, Shcbp1, Bub1, Sgol2, Cdc25b, Mcm2, Mcm4, Mcm5, Mcm7, Myc, Spc24, Kif24, Kif11, Ndc80, Epr1, Ttk, Mybl2, Plk1, Kif14, Cdkn2c, E2f2, Aurkaps1, Pttg1, Cit, Mast1, Melk, Psrc1, Casc5, Mcm6, Chaf1, Gmnm, Cdc7, Spbc25, Chek1.

Having defined the characteristics of the cell cycle-mitosis network in multiple mouse and rat tissues of different situations as prescribed by the MCV process described above (additional MCV steps are to be presented below), studies next evaluated the validity of the LFNR principle as applied to the cell cycle-mitosis network.

It has been shown that the characterization of gene expression traits for cis eQTGs of eQTLs in segregating mouse populations provides multiple lines of evidence that greater than 70% of cis QTGs have documentable gene expression effects. [See, Doss S, Schadt E E, Drake T A, Lusis A J. Cis-acting expression quantitative trait loci in mice. Genome Res 15:681-91, 2005.]. Based on actual observations by the inventor, of the approximately 775 cell cycle and mitosis genes that exist, approximately 10% show “cis” regulation with tissue specificity or variability

Therefore, to further validate the cell cycle-mitosis network using the combined MCV process and LFNR platform described in Section 1, studies were performed to determine if genes with a shared linked function are evident in the eQTG gene sets associated with the eQTLs for the cell cycle-mitosis network. Table 2 presents results from this analysis. The data specifically present representative results via an analysis of four different tissues and two sexes. The characteristics of the eQTL of interest are shown as are the corresponding total numbers of eQTGs and the total numbers of cis eQTGs. Having thereby defined the cis eQTG gene sets, the cis eQTG are next analyzed to determine if each was enriched in genes with a functional linkage as demanded by the LFNR principle.

TABLE 2
Cell Cycle-Mitosis Network eQTLs and Candidate eQTGs

| | Chromosome | | Total |
|---|---|---|---|
| | containing eQTL | Total eQTG | “cis” eQTG |
| Tissues | at (megabases) | Candidates | Candidates |
| Liver BXD (F) | 2 (100-135) | 163 | 25 |
| Liver BHHBF2 | 11 (102-116) | 196 | 10 |
| | 17 (12-28) | 270 | 21 |
| Lung BXD | 9 (110-125) | 119 | 22 |
| Spleen BXD (UWA) | 15 (85-100) | 90 | 9 |
| Adipose BHHBF2 (F) | 4 (45 -70) | 117 | 8 |
| | 6 (35-50) | 112 | 17 |
| Adipose BHHBF2 (M) | 2 (4-24) | 101 | 5 |
| | 8 (88-100) | 81 | 19 |
| 6 Specimens | 9 Chromosome | 1249 | 146 |
| | Locations | | |

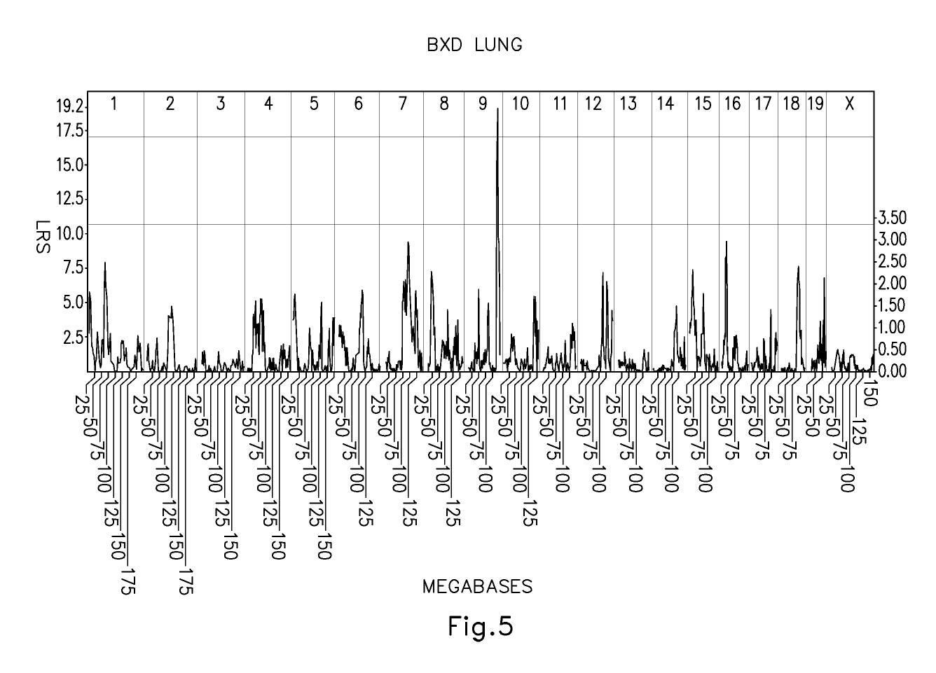

FIG. 5 through FIG. 8 present the characteristics of representative eQTLs on which the data in Table 2 were compiled. They are provided to illustrate that in different situations the eQTLs for the cell cycle-mitosis network are indeed distinct. This dictates that the eQTGs and associated LNFRs for the cell cycle-mitosis network in distinct situations must also be distinct.

The LFNR principle was next tested with respect to the cell cycle-mitosis network based on the 146 candidate cis eQTGs listed in Table 2.

In one embodiment, the Linked Function Network Regulator (LFNR) principle and LFNR platform provides a unique approach to define the best set of candidate genetic regulators (eQTGs) for a network by identifying therein a subset of cis eQTGs that have a linked function in sets of such a network in various species, sexes, tissues, cells, and situations.

In another embodiment, the cell cycle-mitosis network and its genetic regulators are used to validate the LFNR principle. In the present invention, such an analysis was performed on the above best six datasets.

Note that for each dataset, the cis candidate eQTGs that are associated with significant eQTLs are tabulated in the following listing. The parenthetic statements associated with the description of the dataset show the total number of cis candidate eQTGs and whether the dataset is from females (F), males (M) or both sexes (BS). Additional parenthetic terms are included in certain situations to define alternate abbreviations for certain genes.

BXD Liver-F (25): Lmo2, Ltk, Mga, Sinn (Zfp106), Slca2, Mmrp19 (Apip), Ivd, Itpka, Rgap1 (1Racgap1), PLA2G4B Pla2g4b (Pa24b), Capn3, Cnndbp1 (Gcip), Catsper2, Mfap1, B2m, Sdh1 (Sdhb), Slc30a4, Cops2 (Alien), Mpped2, Fibin, Fam82a2, Gchfr, Tmem87a, Haus2 (Cep27), Adal.

BHHBF2 LIVER-F (30): Prkar1a, Wtap, Pkmyt1, Ccnf, Tsc2, Acbd4 Kpna2, Helz, Cog1, Cd300a, Rnf157, St6gainc2, Syngr, Map3k4, Pnldc1, Acat2, Tceb2, Zfp598, Gfer, Tbl3, Traf7, Rps2, Hs3st6, Nubp2, Ift140, Telo, Gnptg, Wfikkn1, Decr2, Tmem8.

BXD LUNG-BS (22): Rmbs3, Limd1, Clasp2, Champ (Mov1011), Ifrd2, Ccdc72, Tmem7, Crtap, Glb1, Acaa1b, Acaa1, Rpl14, Sec22l3, Deb1, Nktr, Hig1, Ccbp2, Ccr1, Ccr2, Ccr5, Ulk4, Tmem103.

BXD SPLEEN-F (9): Epas (Rapgef3), Ttll12, Arsa, Kif21a, Pp11r, Tmem106c, Senp1, Adcy6, Accn2.

BHHBF2 ADIPOSE TISSUE-F (25): Hoxa2, Smc2, Tbxas1, Rab19, Ndufb2, Gstk1, Zfp467, Rarres2, Zfp775, Tmem176b, Gpnmb, Cdcc126, Mpp6, Dfna5h, Skap2, Hibadh, Plekha8, Gars, Mcart1, Txndc4, Ecm29, Gbg10, Bspry, Alad, Zfp618.

BHHBF2 ADIPOSE TISSUE-M (24): Gadd45gip1, Usp38, Elmod2, Cd97, Asf1b, Trmt, Lul1, Rad23a, Farsia, Gcdh, Fbxw9, Vps35, Mmp2, Capns2, Pllp, Ciapin1, Gpr97, Gins3, Ndrg4, Usp6n1, Ptpla, Scl339a12, Armc3, Lcn4.

In one embodiment, published abstract analyses are performed on this set of candidate cis eQTGs using PubMed, Genecard, NCBI Resources—Gene and other online tools to document the function of all 146 candidate cis eQTGs. In certain situations a detailed review of the actual referenced scientific paper was also performed when review abstracts appeared to be equivocal.

In an associated embodiment, the above review of the scientific literature related to each candidate eQTG is analyzed to determine if any gene set with a linked function can be identified. The outcome of those analyses validates the LFNR principle as defined in Section 1. In the present invention, the results presented in the next listing establish that the only linked function of the LFNRs for the cell cycle-mitosis network is cell cycle and mitosis.

For the following tissues, candidate cell cycle and mitosis LFNRs are:

LIVER-BXD: Mga (CELL CYCLE)-CHR 2-LRS=13 to 68; Ccndbp1 (CELL CYCLE)-CHR 2-LRS=32; Mfap1 (MITOSIS)-CHR 2-LRS=68; Cops2 (CELL CYCLE)-CHR 2-LRS=15; Mpped2 (CELL CYCLE)-CHR 2-LRS=27; Haus2 (MITOSIS)-CHR 2-LRS=12.

LUNG-BXD: Rbms3 (CELL CYCLE)-CHR 9-LRS=11; Clasp2 (MITOSIS)-CHR 9-LRS=98; Champ (CELL CYCLE)-CHR 9=LRS=11; Nktr (MITOSIS)-CHR 9-LRS=113.

SPLEEN-BXD: Epac (MITOSIS)-CHR 15-LRS=14; Senp1 (CELL CYCLE) CHR 15-LRS=28.

LIVER-BHHBF2: Wtap (MITOSIS)-CHR 17-LRS=400; Pkmyt1 (CELL CYCLE)-CHR 17-LRS=25; Ccnf (CELL CYCLE)-CHR 17-LRS=>165; Nubp2 (MITOSIS)-CHR 17=LRS >45; Tsc2 (CELL CYCLE)-CHR 17-LRS=18 Gfer (CELL CYCLE)-CHR 17-LRS=>270.

ADIPOSE TISSUE-BHHBF2: Smc2 (MITOSIS)-CHR 4-LRS=16.5; Hoxa2 (MITOSIS)-CHR 6-LRS=17.5; Gadd45gip1 (CELL CYCLE)-CHR 8-LRS=48.5; Asf1b (CELL CYCLE)-CHR 8-LRS=25; Ciapin1 (CELL CYCLE)-CHR 8-LRS=168; Ndrg4 (CELL CYCLE)-CHR 8-LRS=52; Usp6n1 (MITOSIS)-CHR 2-LRS=18.

In order to confirm that cell cycle-mitosis genes of the present invention were indeed enriched in the cis subset of total candidate eQTGs, two approaches may be used to determine the degree of actual enrichment.

The first method calculates the enrichment based on observed versus expected values using a range of the total number of cell cycle-mitosis genes that exist in the genome as reported in various publications that range from about 480 to about 800 and about 15% cis frequency as an average published and observed frequency. Based on these calculations, the enrichment in the present case was determined to be greater than about 350%.

To substantiate that level of enrichment, a second method was used that involved the actual measurement of cis cell cycle-mitosis gene frequency using megabase segments that were comparable in size to the eQTLs of each dataset. The enrichment observed using this method was greater than about 450%, thus confirming that the LFNR principle and the finding that small sets of cis cell cycle-mitosis genes represent prime candidate eQTGs to regulate the cell cycle-mitosis network of the present invention.

In one embodiment, the LFNR principle does not require that the linked function designation must always reflect the function of the actual network being regulated. In another embodiment, it is anticipated that in the future systems genetics networks with a specific function will be identified in which the genetic regulators of that network will have a totally distinct but linked function that is shared by all the genetic regulators for that network in various species, strains, sexes, tissues, cells and situations.

However, the information presented in this section, establishes that the cell cycle-mitosis network and its genetic regulatory mechanisms satisfy all LFNR platform requirements and therefore validates the value of the LFNR platform.

In this embodiment, the present invention provides that the MCV process is thereby validated by the data on the cell cycle-mitosis network and that the LFNR principle is also validated by the date on the cell cycle-mitosis network eQTLs and cis eQTGs using primarily mouse—but can also include rat datasets—involving different sexes and different tissues.

In another embodiment, the present invention provides for methods and process for use of mouse cell model systems to establish that specific RNAi, drugs or combinations that target specific LFNRs or combinations thereof that have the potential to impact LFNR expression and/or function and thereby influence cell cycle-mitosis network characteristics and thus further validate the functional role of such LFNRs as regulatory factors for the cell cycle-mitosis network.

To further validate the MCV process with respect to the cell cycle-mitosis network an additional series of requirements that must therein be fulfilled. Therefore the following embodiments are presented:

1. Establish that the cell cycle-mitosis network of covariate expressed genes exists in two or more tissue or cell types.

In another embodiment, the MCV process comprises a step of establishing that the specific network of covariate expressed genes with correlation coefficients >0.5 exists in multiple tissues or cell types. In another embodiment, the MCV process comprises using a recombinant inbred mouse system and other related animal populations.

Concerning the cell cycle-mitosis network of the present invention, Table 1 documents that this network exists in multiple tissues of mice and rats.

A seminal finding is that cell cycle-mitosis networks can have different compositions of genes in different tissues. For example, the cell cycle-mitosis has a distinct composition of genes in the liver of BXD mice versus the livers of BHBHF2 mice. Another embodiment in this regard, is that the cell cycle-mitosis network has distinct compositional characteristics in all four strain and sex possibilities so that BXD males, BXD females, BHHBF2 males and BHHBF2 females are all distinct.

Another key characteristics of the cell cycle-mitosis network that actually exceeds the requirement of the MCV process is that differences in the characteristics of the cell cycle-mitosis network exist between sexes in additional tissues including the liver and adipose tissue as documented in Tables 1 and 2 and by the following embodiment.

In another embodiment, even though the cell cycle-mitosis network within the liver of female and male BXD mice are distinct, they do contain 28 identical network members when Cdc20 is used as the “key” network gene of interest. These 28 cell cycle-mitosis genes of the network are: Cdc20, Aurka, Ccna2, Cenpe, Cdca8, Ncapg, Prc1, Plk1, Mki67, Mcm5, Ccnb2, Cdc2, Aurkb, Spag5, Birc5, Cenph, Racgap1, Sgol1, Kif20a, Ccd25c, Cdca5, Mphosh1, Nuf2, Cenpf, Nek2, Nusap1, Tpx2 and Ube2c. As described in Section 1, up to 50% of the components of the cell cycle-mitosis network can be shared in various situations.

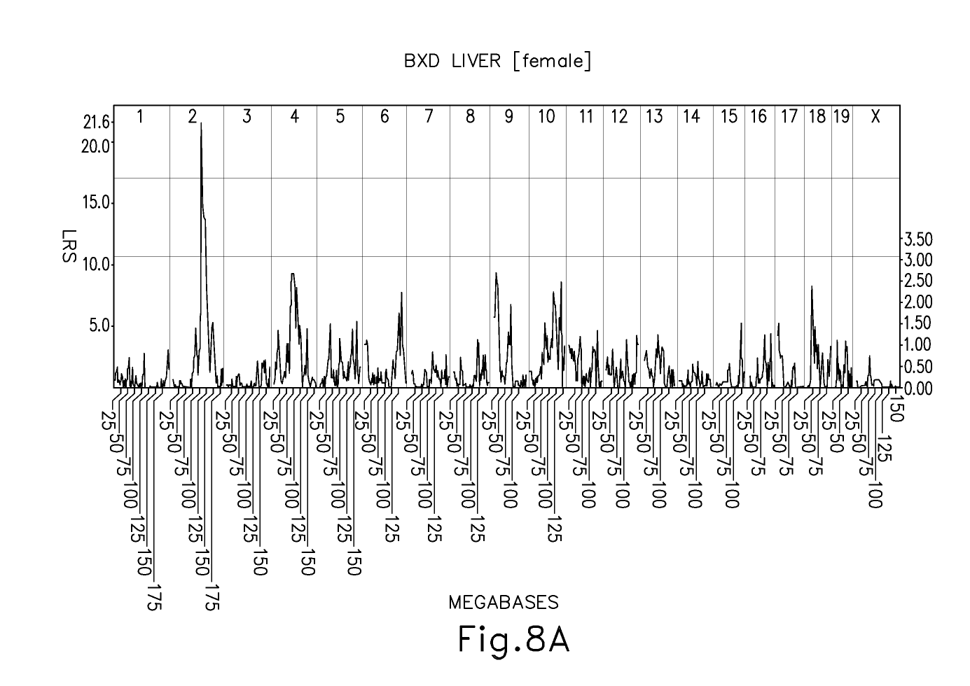

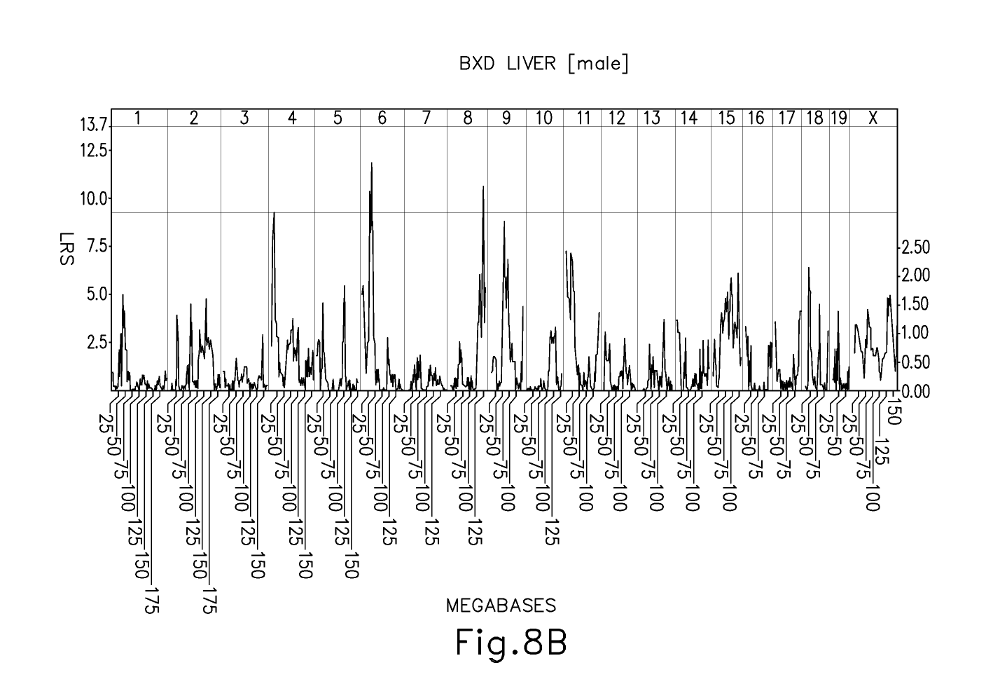

FIG. 8a and FIG. 8b document the cell cycle-mitosis network sexual dimorphism that exist in BXD livers. When eQTL mapping is performed on the cell cycle-mitosis network comprised of the 28 identical genes in females and males, totally distinct eQTL patterns are evident.

FIG. 8a and FIG. 8b show that in females there is a single significant chromosome 2 eQTL for the special 28 gene network as described above whereas in the liver of males it is polygenetic with suggestive eQTLs on chromosomes 4, 6, and 8.

2. Establish that the cell cycle-mitosis network exists in two or more databases developed by different laboratories and/or investigators.

The cell cycle-mitosis network of the present invention has been identified and characterized in specimens prepared by investigators at multiple different institutions including: 1) the University of Tennessee Health Science Center, 2) the University of North Carolina, 3) the University of California—Los Angeles, 4) Helmholtz Zentrum für Infektionsforschung GmbH in Germany and 5) Rosetta Inpharmatics, Seattle, Wash., among others. All these databases are available via open access in GeneNetwork (www.genenetwork.org).

3. Establish that the cell cycle-mitosis network can be replicated in databases developed using two or more different microarray technologies and platforms.

The cell cycle-mitosis network of the present invention has been identified and characterized in specimens prepared using the following such technologies and platforms specifically involving the parenthetic examples that are available via open access in GeneNetwork: (a) Agilent (UNC Agilent G4121A Liver LOWESS Stanford (January 6) Both Sexes); (b) Affymetrix (HZI Lung M430v2 (April 8) RMA); and (c) Illumina (GSE9588 Human Liver Normal (March 11) for both Sexes).

4. Establish that the cell cycle-mitosis network can be reproduced in databases developed using at least two different microarray data normalization systems, i.e., MASS versus RMA.

The cell cycle-mitosis network of the present invention has been identified and characterized in specimens using the following microarray data normalization systems specifically involving the parenthetic examples openly available in GeneNetwork: (a) MASS (SJUT Cerebellum October 3); (b) RMA (HZI Lung April 8 and NCI Mammary April 9); and an (c) Miratio (UCLA BHHBF2 Liver Male).

5. Establish that the cell cycle-mitosis network exist in databases developed using different animal and strains, i.e., BXD mouse strains and various F2 mouse populations, plus different animal species, such as, rats.

The cell cycle-mitosis network of the present invention has been identified and characterized in specimens prepared using different animal and strains, plus different animal species specifically involving the parenthetic examples openly available in GeneNetwork: BXD mice (UNC Agilent G4121A Liver LOWESS Stanford January 6 and others), BHHBF2 mice (UCLA BHHBF2 Liver Male Only), and HXB/BXH rats (MDC/CAS/UCL Liver December 8).

6. Establish that the cell cycle-mitosis network shows one or more suggestive or significant eQTLs in at least the most significant examples of all the studied situations.

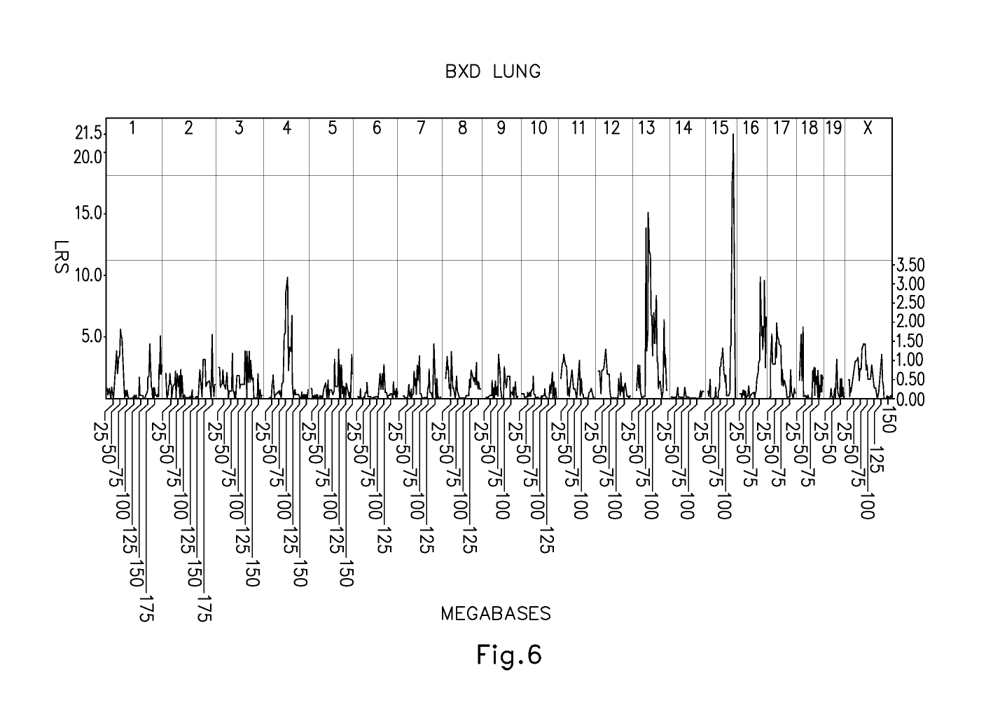

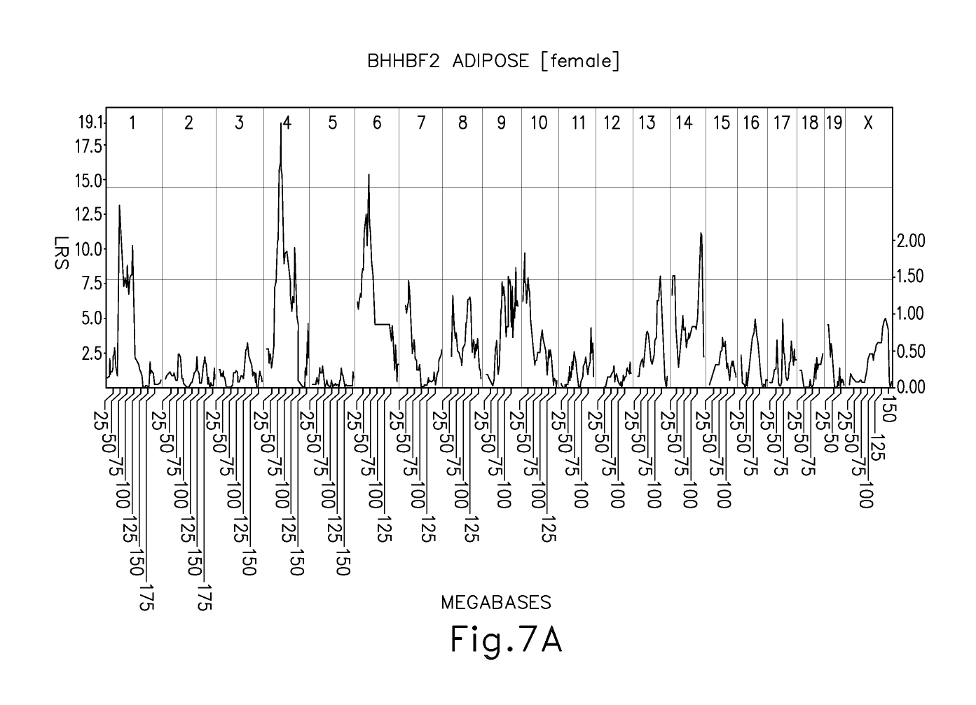

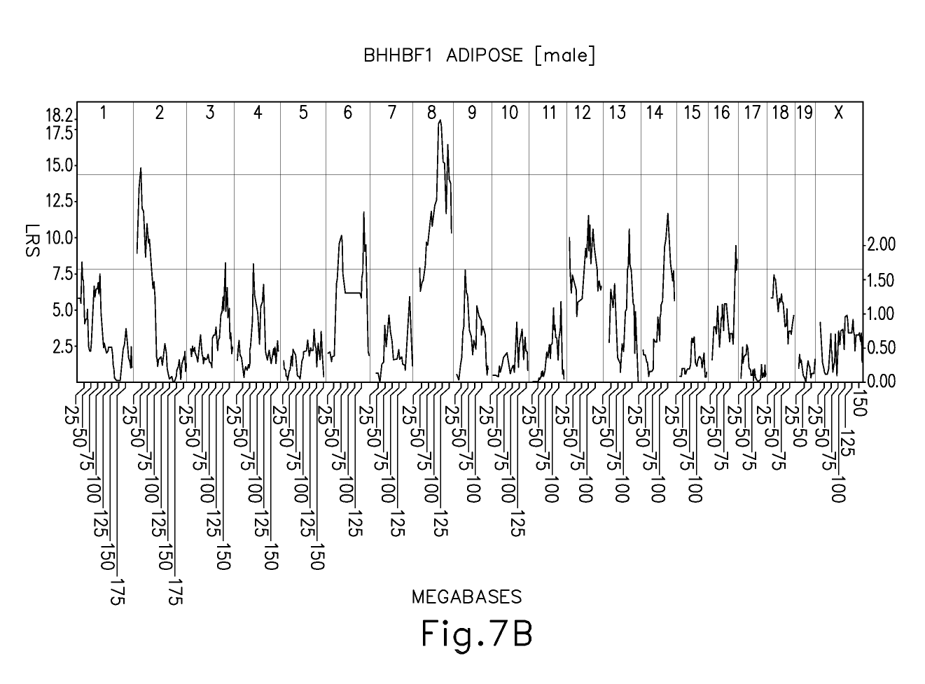

FIG. 5 to FIG. 8 document that eQTLs for the cell cycle-mitosis network exist in multiple studies tissues including BXD liver (male and female), BHHBF2 adipose tissue (male and female), BXD spleen, and BXD lung. FIG. 5 is a chart showing the chromosome 9 eQTL for BXD lung cell cycle-mitosis network of genes that show covariant expression with Cdc20. FIG. 6 is a chart showing the eQTLs for BXD spleen cell cycle-mitosis network for genes that show Cdc20 expression covariance. The chromosome 15 eQTL has high significance. FIG. 7A is a chart showing the BHHBF2 adipose tissue cell cycle-mitosis network eQTL with sexual dimorphism in females. FIG. 7B is a chart showing the BHHBF2 adipose tissue cell cycle-mitosis network eQTL with sexual dimorphism in males. FIG. 8A shows that the BXD female liver cell cycle-mitosis network has a chromosome 2 eQTL. FIG. 8B shows that the cell cycle-mitosis network in BXD male liver has eQTLs that are polygenetic with suggestive eQTLs on chromosomes 4, 6, and 8.

7. Establish that the cell cycle-mitosis network exists only in tissues and/or cells that are physiologically relevant (proliferative) and not in tissues and cells that are not physiologically correct (non-proliferative), (negative control).

The brain, which is essentially non-proliferative, shows no cell cycle-mitosis networks in representative samples that include: 1) the human whole brain database (GSE5281 Human Brain Normal July 9 RMA) when the cell cycle-mitosis network was searched for in the present invention by analyzing of the top 500 expression covariants using Cdc20 as the key gene of interest combined with gene ontology analysis to search for a common function, 2) the BXD whole brain database (UCHSC RMA November 6), 3) the BXD cerebellum database [SJUT MASS October 3), and 4) the BXD hippocampus database (Consortium RMA November 6). The latter three tissue were searched for the cell cycle-mitosis network in the present invention by analysis of the top 100 and 500 expression covariates using Cdc20 or Aurora A as key gene of interest combined with gene ontology analysis (WebGestalt-GoTree).

In another embodiment, steps 1 through 7 for the cell cycle-mitosis network and its regulatory LFNRs (QTLs and QTGs) are accomplished using a bioinformatics computer system, GeneNetwork (genenetwork.org).

### Section 4: Special Data on the Cell Cycle-Mitosis Network in an Animal Cancer Specimen

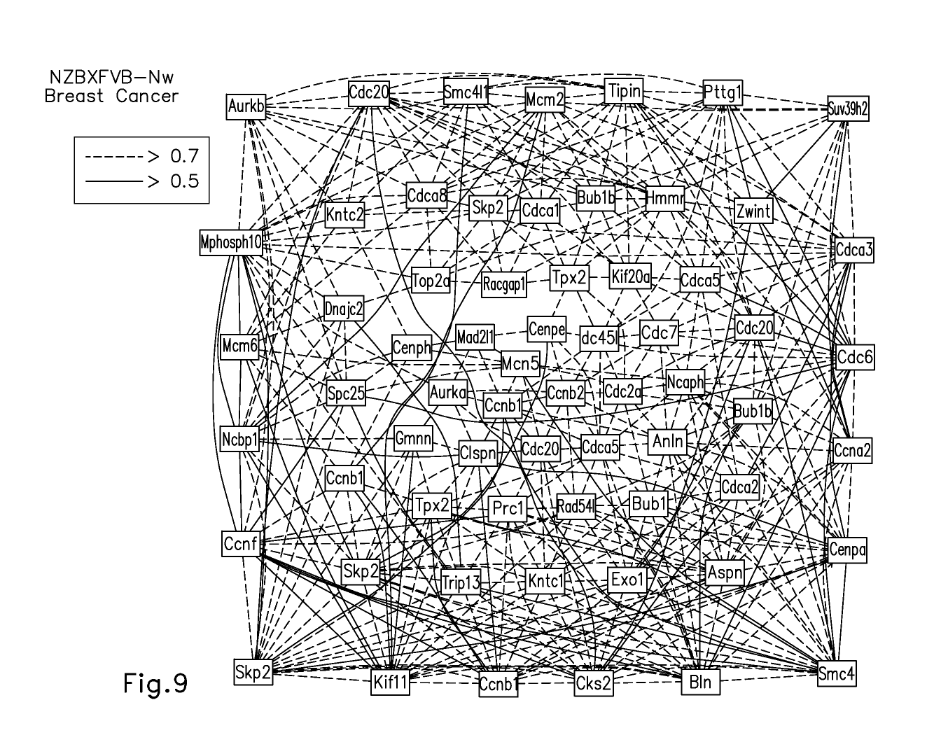

Although not a requirement of the MCV process or LFNR platform of the present invention, an additional step has been performed to establish that a cell cycle-mitosis network exists in cancer tissues. Because cell cycle and mitosis lesions are a hallmark of carcinogenesis, the mouse breast cancer database designated NCI Mammary M430v2 (April 9) RMA, which is openly available in GeneNetwork, was used to confirm existence of the cell cycle-mitosis network of the present invention (FIG. 9). A search of the NZB×FVB−Nw breast cancer database for the top 500 genes that show expression covariance with Cdc20 as the key gene of interest documented 55 network components with a common cell cycle or mitosis function. Analysis of these genes using gene ontology methods demonstrates that the cell cycle-mitosis network has a very high significance (p=5.60×ê−27).

Detailed analysis of the characteristics of the mouse breast cancer cell cycle-mitosis network shows that the vast majority of the genes showing covariate expression with Cdc20 have correlation coefficients >0.7. Furthermore, the data show that a subset of twenty four (24) breast cancer cell cycle-mitosis network genes show correlations coefficients of >0.9 which is extraordinary.

Gene ontology data based on the characteristics of the cell cycle-mitosis network in these breast cancer specimens also establish a significance that varies from 4.23×ê−26 to 2.20×ê−32 depending on which gene ontology characteristic is chosen.

These findings show that the cell cycle-mitosis network exists in cancer tissue and that such cancer networks of cell cycle and mitosis gene can also have distinct characteristics.

The cell cycle-mitosis network in animals shows species, strain, sex, tissues, cell type and situation specificity. Therefore, the same cell cycle-mitosis network characteristics should exist in humans wherein the network should show race, sex, tissue, and cell type specificity. Analysis of normal specimens of the human tissues and/or cells from patients with disease proclivities has the potential to generate insights into disease prevention. In contrast, for cancers of various types and causes from patients from different races and sexes, the cell cycle-mitosis network and its genetic regulators (LFNRs) will need to be defined in specimen populations of each cancer specificity so that the associated specific LFNR will have the potential to serve as prime targets for a new class of cancer drugs. In a further embodiment, such studies will require that comparable analysis be performed on genetic variation panels of control and disease specimens from individual human cancer types with race, sex and tumor tissue type specificities.

Section 5: Procedure to Translate Cell Cycle-Mitosis Networks and their LFNRs from Non-Human Animals to Humans and Definition of the Characteristics of Human Cell Cycle-Mitosis Networks and their LFNRs Human Liver Specimens.

Based on all the evidence presented herein concerning the cell cycle-mitosis network and its genetic regulators (LFNRs) that have been discovered and characterized using non-human animal models, the translation of those findings to the human situation has been established using a human liver cohort dataset that is openly available in GeneNetwork.

The liver dataset in GeneNetwork that has been used for this purpose consists of gene expression data derived from 427 Caucasian individuals as defined in the database designated GSE9588 Human Liver Normal (March 11) Both Sexes. DNA samples were genotyped on the Affymetrix 500K SNP and Illumina 650Y SNP genotyping arrays, representing a total of 782,476 unique single nucleotide polymorphisms (SNPs). [See: Schadt E E, et al., Mapping the genetic architecture of gene expression in human liver. PLoS Biol. 6:e107 (2008)].

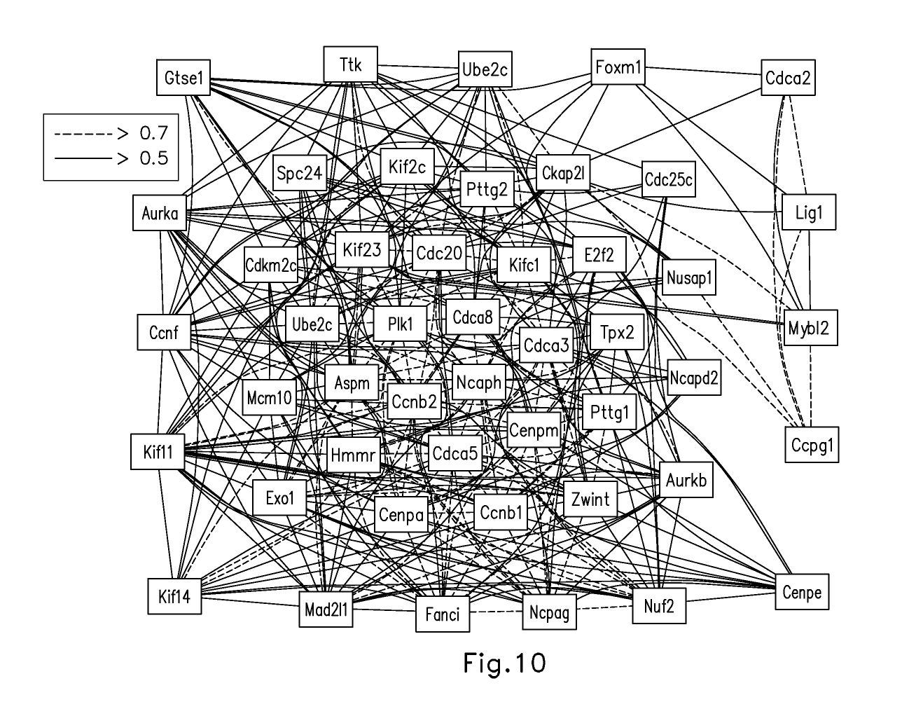

The human liver cell cycle-mitosis network was optimally identified by searching for gene expression covariates with Cdc20 that have a correlation coefficient of greater than 0.5. In the human liver database for both sexes, a total of 47 cell cycle-mitosis network genes with correlation coefficients greater than 0.5 using Cdc20 as the key gene of interest as shown in FIG. 10. Table 3 shows this data and that when the human liver datasets are separated into separate male and female components adequately high significance is retained even thought the size of the cell cycle-mitosis network is somewhat smaller that for the data from both sexes.

TABLE 3
Human Liver Cell Cycle - Mitosis Network Characteristics

| | Total number | Highest |
|---|---|---|
| | of genes in each cell | network |
| Tissue | cycle-mitosis network | significance |
| Human liver - both sexes | 47 | p = 2.18e\hat{ }−20 |
| Human liver - male | 24 | p = 3.71e\hat{ }−11 |
| Human liver - female | 40 | p = 5.41e\hat{ }−10 |

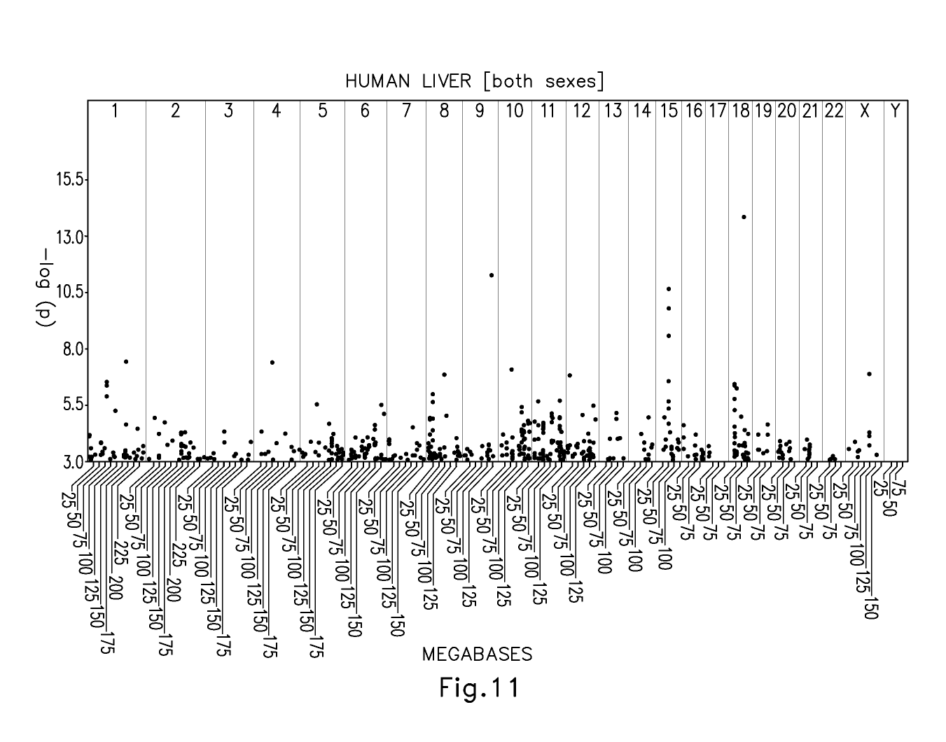

When a GWAS SNP analysis is performed concerning the network of 47 covariate cell cycle-mitosis genes of the Caucasian human liver of both sexes, the results of mapping outcomes show that chromosomes 9, 15 and 18 display the most significant GWAS SNPs with values greater than 8.0−log P. (See FIG. 11). For this analysis, winsorization (partial) was performed on two of the 427 individual dataset because they were outliers.

Concerning the statistics involved in GWAS SNP analysis, GWAS SNPs at −log(P)=>4.0 are considered to be of possible significance; GWAS SNPs with −log(P)=>5.0 are considered to be of probable significance and GWAS SNPs with log(P)=>8.0 are considered to be of definite significance. These criteria are identical to those stated previously in this document.

The GWAS SNP on chromosome 18 is not associated with a gene and is not considered to be of particular relevance. In contrast, the GWAS SNP on Chr 9 is associated with the gene designated Astn2 (rs7026807) with a value of 11.2248−log P and the six GWAS SNPs on chromosome 5 are associated with the gene designated Aro1 (rs16964201) with values of 5.6232−log P, (rs1865803), 9.7340−log P, (rs17647719), 8.5400−log P, (rs7167343), 10.6171−log P, (rs12594203), 5.3004−log P, (rs999480), 4.6249−log P, and 6.5117−log P (rs8031463).

As used herein, the gene Aro1 is also known as CYP19A1 or cytochrome P450, family 19, subfamily A, polypeptide 1 or CYP19 CYAR, ARO, CPV1, P-450AROM, aromatase, cytochrome P450, subfamily XIX, Cytochrome P-450AROM, estrogen synthase, CYPXIX, EC 1.14.14.1, cytochrome P450 19A1, or estrogen synthetase. As used herein, the parenthetic terms designated (rs) related to the GWAS SNP markers of interest associated with a specific gene.

Since studies on the cell cycle-mitosis network in the liver of BXD mice demonstrated definitive evidence of sex dimorphism, before proceeding to further analyze all the additional GWAS SNP data in human liver with statistical significance of greater than −log P<4.0, the human liver cohort was segregated into male and female subsets that includes 193 males and 234 females for further analysis. To optimize the data, outliers were winsorized.

### Section 6. Detailed Information on the Cell Cycle-Mitosis Network for Human Caucasian Female Liver and its GWAS SNPs as LFNRs.

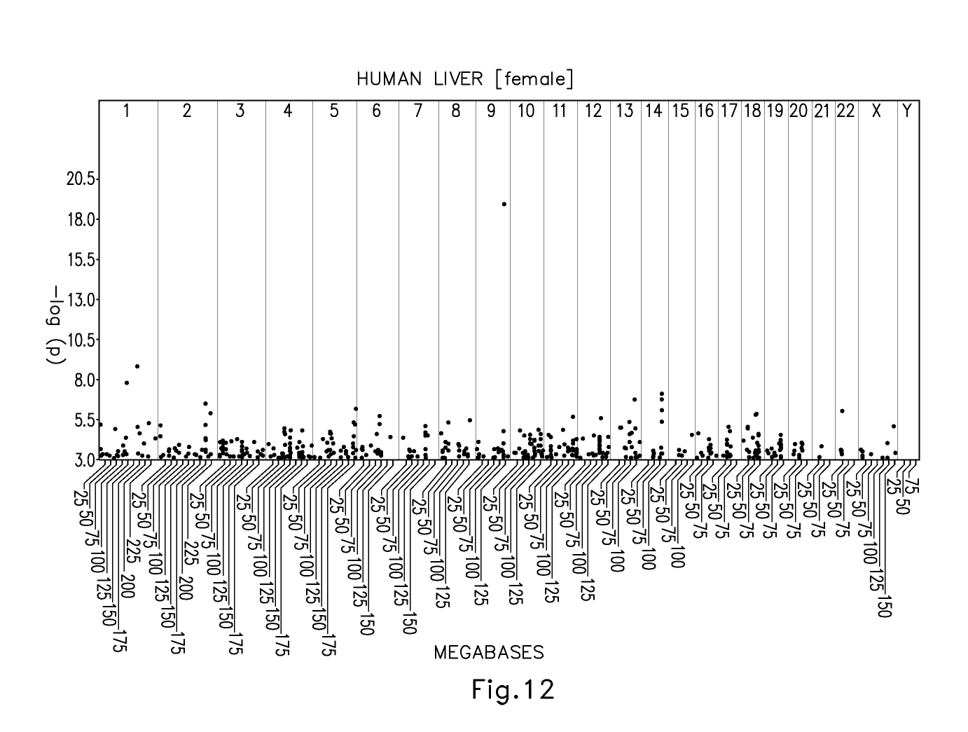

FIG. 12 shows the results for the Caucasian female cell cycle-mitosis network dataset, specifically that the chromosome 9 GWAS SNP for the Astn2 gene is female specific. In addition, the data show that there are many additional GWAS SNPs greater that 4.0−log P to be considered related to the cell cycle-mitosis network in this dataset.

An analysis of the data establish that for the Caucasian female cell cycle-mitosis network, there are two genes containing GWAS SNPs that have a significance of >8.0−log(P). They include Astn2 at 18.87−log P (rs7026867), and Tbx19 at 8.74−log P (rs2075976) and 4.98−log P (rs11770655).

There are an additional nine genes containing GWAS SNPS that have a significance of from 5.0 to 8.0−log P. They are: Piwil3, Abca12, Bach2, Cxadr, Fgf18, Nrg1, Ush2a, Nsd1, Prdm16. Of these three have a function that can be linked to the cell cycle and/or mitosis. They include: Cxad at 5.29−log P (rs211953); Nrg1 at 5.21−log P (rs2347510) and Prdm16 at 5.07−log P (rs17390062).

Furthermore, there are 27 genes containing GWAS SNPs with a level of significance of from 4.0 to 5.0−log(P) in the Caucasian female cell cycle-mitosis network. These include: Hrh4, GpcS, Nrap. Rps3, Lbra, Dapp1, Sp2, Lhfpl3, Astn2, Sipa1l3, Gfm2, Csmd1, Cenph, Galnt4, Prkg1, Tmtc3, Cdk2ap1, Nell1, St8sia5, Rerg, Fam169a, Smyd3, Ntm, Robo2, Accn1, Cyp2c8, Plcl2, Crybg3. Of these, 5 genes containing GWAS SNPs can be linked to cell cycle and/or mitosis: They include: Dapp1 [Bam32] at 4.71−log P (rs767652); Cenph at 4.53−log P (rs100192); Cdk2ap1 at 4.33−log P (rs3759114); Nell1 at 4.32−log P (rs16907322) and Symd3 at 4.24−log P (rs4654179).

The following listing describes each gene that contains a GWAS SNP of interest and its relevance to the cell cycle and mitosis. In this listing the parenthetic word provides insight as to whether a particular gene is linked to the cell cycle and/or mitosis.

Astn2—(maybe)—regulates the cell surface expression of various proteins and receptors via clathrin-mediated endocytosis which can be modulated during mitosis.

Tbx19—(probable)—in the developing pituitary the absence of Tbx19 results in the accumulation of noncycling precursor cells that co-express p57^{Kip2} and p27^{Kip1} which are cell cycle progression inhibitors. Double knockout mice for p27^{Kip1} and p57^{Kip2} have been established to be defective cell cycle exit for differentiation.

Cxadr—(certain)—can elicit a negative signal cascade to modulate cell cycle regulators inside the nucleus of bladder cancer cells in association with the accumulation of p21 and hypophosphorylated Rb1. The fact that Cxadr can be associated with E-cadherin and p53 in the urothelium also suggests that it can impact the cell cycle.

Nrg1—(probable)—acting thru its ERBB4 receptor, the injection of NRG1 in adult mice induces cardiomyocyte cell-cycle activity and promotes myocardial regeneration.

Prdm16—(probable)—is a transcription factor that regulates a remarkable number of genes that, based on knockout models, both enhance and suppress human stem cell function, and affect quiescence, cell cycling, renewal, differentiation, and apoptosis.

Dapp1 (Bam32)—(certain)—promotes B lymphocyte entry into the G1 stage of the cell cycle and regulates the downstream expression of p27^{kip1} so that Dapp1-knockout B lymphocytes appear to be able to enter into early G₁-phase but inefficiently progress to later G₁ stages that promote S-phase entry.

Cenph—(certain)—has an important role in the architecture and function of the human kinetochore complex. In CENP-H knocked-down cells, severe mitotic phenotypes like misaligned chromosomes and multipolar spindles are evident but mitotic arrest does not result. Cenph also regulates the incorporation of Cenpa into the kinetochore and can interact with Trim36 to delay cell cycle progression.

Cdk2ap1—(certain)—is a cell cycle regulator that can function as a growth suppressor. Its impact on the cell cycle has recently been mechanistically linked to epigenetic control processes.

Nell1—(certain)—the binding of the growth factor Nell1 to APR3 significantly inhibits proliferation of osteoblasts by increasing the down-regulation of Cyclin D1 in association with NELL-1 and APR3 co-localized on the nuclear envelope.

Symd3—(certain)—a histone methyltransferase that plays an important role in transcriptional regulation including genes involved in the control of cell cycle (e.g., CyclinG1 and CDK2). Its down-regulation induces G₁-phase cell cycle arrest.

The fact that the most significant GWAS SNP-associated gene for the cell cycle-mitosis network in human Caucasian female liver, i.e., Astn2, is not absolutely proven to be linked to the cell cycle-mitosis network limits its potential to use it as potential candidate drug target until many additional studies on the Astn2 are reported. Alternately, additional studies could be performed on the GWAS SNP-associated genes that have a stronger linkage to the cell cycle and mitosis, especially Tbx19.

These results validate the LFMR principle and LFNR platform by confirming that GWAS SNPs for the cell cycle-mitosis network are enriched in cell cycle and mitosis genes as predicted from all the prior data derived from studies in non-human animals. More specifically, five of the 11 genes containing GWAS SNPs for the cell cycle-mitosis network with >5.0−log P values are implicated or proven to be linked to the cell cycle and/or mitosis. An additional five of 23 genes containing GWAS SNPs for the cell cycle-mitosis network with >4.0−log P are also implicated or proven to be linked to the cell cycle and/or mitosis. Therefore 10 of 34 or ˜30% of these GWAS SNP for the cell cycle-mitosis network are implicated or proven to be linked to the cell cycle and/or mitosis genes. This represents a significant enrichment since known cell cycle-mitosis genes comprise only 3 to 5% of all genes encoded by the human genome depending on the stringency of the criteria to designate a gene of interest to be linked to the cell cycle and/or mitosis.

### Section 7. Detailed Information on the Cell Cycle-Mitosis Network for Human Caucasian Male Liver and its GWAS SNPs as LFNRs.

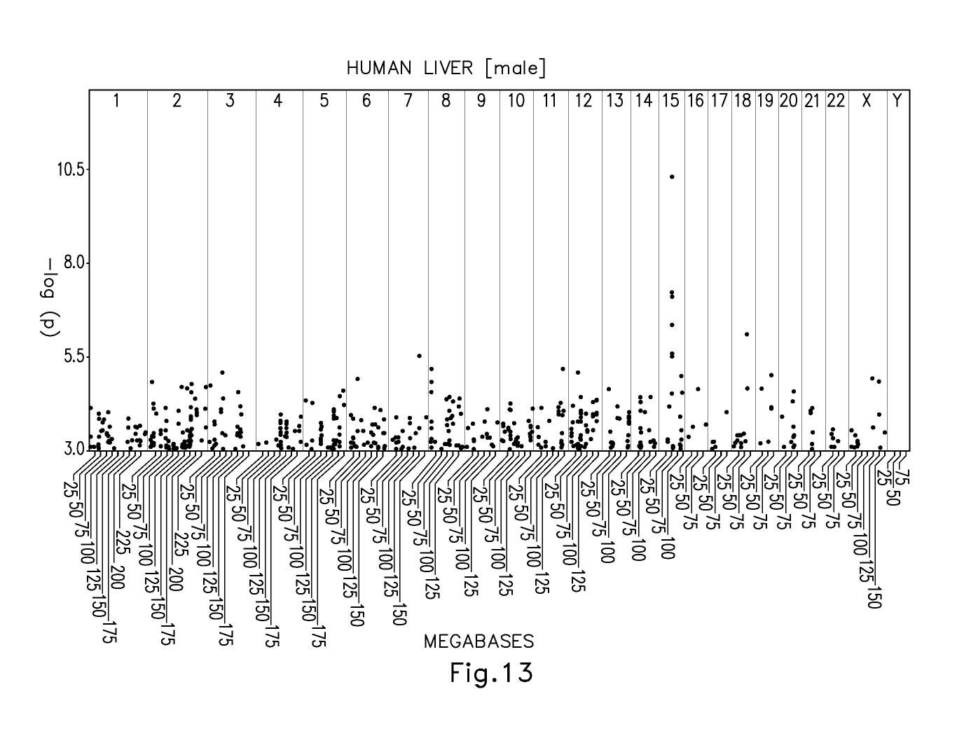

FIG. 13 shows the results for the Caucasian human male cell cycle-mitosis network dataset specifically that the chromosome 15 GWAS SNP for the Aro1 gene is male specific. The data also show that many additional GWAS SNPs greater that 4.0−log P exist.

A detailed analysis documents that there is only one gene with GWAS SNP that has a significance greater than 8.0−log P. This gene is Aro1 at 10.25−log P (rs71677343), plus Aro1has additional GWAS SNPs of 7.19−log P (rs17647719), 7.08−log P (rs999480), 6.32−log P (rs12594203), 5.54−log P (rs8031463), 5.47−log P (rs1865803), and 4.49−log P (rs 16964201).

There are then four male liver genes with GWAS SNPs >5.0−log(P), <8.0−log P. They are: Angpt2, Ncam1, Syt10, Fhit. Of these, there is one GWAS SNP-containing gene that has a function that is linked to the cell cycle and/or mitosis and it is Angpt2 at 5.14−log P (rs2442611), and 4.53−log P (rs2442612).

There are also 20 genes containing GWAS SNPs with significance levels from 4.0 to 5.0−log(P). They include: Nlrp5, Kif6, Pde11a, Grm7, Pask, Unc13a, Wwc1, Ap4s1, Npas3, Hegw2, Ptprg, Ubeq11, Cbln4, Pdgrd, Fbxo32, Rdh13, Tragf3ip1, Adamts19, Aox1, Cntnap5. Of these 4 are GWAS SNP containing genes linked to the cell cycle-mitosis network: Wwc1 at 4.57−log(P) (rs11134509); Npas3 at 4.39−log(P) (rs1953444), 4.28 at −log(P) (rs17100034); Ptprg at 4.35−log(P) (rs1508394) and Traf3ip1 at 4.05−log(P) (rs10915551).

The following listing describes each gene that contains a GWAS SNP of interest and its relevance to the cell cycle and mitosis. In this listing the parenthetic word provides insight as to whether a particular gene is linked to the cell cycle and/or mitosis.

Aro1—(certain)—in human breast cancers aromatase inhibitors repress the expression of −90 genes associated with cell cycle progression, particularly mitosis.

Angpt2—(certain)—induces STATS activation, p21waf expression and increases fraction of cells in G1.

Wwc1—(certain)—phosphoprotein member of the Hippo/SWH signaling pathway whose phosphorylation is regulated in a cell cycle-dependent manner with a maximum in mitosis.

Npas3—(probable)—is aberrantly expressed in greater than 70% of a panel of 433 human astrocytomas and drives progression of astrocytomas by modulating the cell cycle and other cancer phenotype determinants.

Ptprg—(certain)—interactions of PTPRG in the extracellular matrix induce cell arrest and changes in cell cycle status. This is associated with inhibition of pRB phosphorylation through down-regulation of cyclin D1.

Traf3ip1—(probable)—one of a set of 15 genes in the TNF/NF-κ B signaling pathway to impact G₂/M.

There are two important outcomes from the analysis of the Caucasian human male liver data, First, the data extend the validation of the LFMR principle and LFNR platform confirming that GWAS SNPs for the cell cycle-mitosis network are enriched in cell cycle and mitosis genes as predicted from prior data derived from studies in animals. Specifically, six of 25 or about 25% of all the GWAS SNP for the cell cycle-mitosis network are implicated or proven as linked to the cell cycle and/or mitosis genes. This again represents a significant enrichment since known cell cycle-mitosis genes comprise only ˜3 to 5% of all genes encoded by the human genome.

The second and perhaps most important outcome from analysis of the human Caucasian male liver data relates to the potential clinical importance of the Aro1 gene that contains seven GWAS SNPs that have significance of 10.3 to 4.5−log P.

### Section 8. The Human Caucasian Male Liver Aro1 Cell Cycle-Mitosis Network GWAS SNPs and its Clinical Relevance to the Prevention of Hepatocellular Carcinoma (HCC) in High-Risk Caucasian Human Males.

The present invention now provides methods of preventing in high-risk Caucasian human males using aromatase inhibitors that target the Aro1 gene product, which is the GWAS SNP (LFNR) of highest significance for the cell cycle-mitosis network of the human population.

Aro1 has been proven as linked to the cell cycle and mitosis in studies using human specimens in many published papers. (see, Miller W R, Larionov A, Renshaw L, Anderson T J, White S, Hampton G, Walker J R, Ho S, Krause A, Evans D B, Dixon J M. Aromatase inhibitors—gene discovery. J Steroid Biochem Mol Biol. 106: 130-42, (2007)).

Protocols of the above referenced paper involved RNA extracts from breast cancer biopsies taken before and after 10-14 days of treatment for use in microarray analysis. Early changes in gene expression were identified by comparing paired tumor core biopsies taken before and after 14 days treatment in 58 patients. The results established that the expression of 91 genes were down-regulated and that these genes were primarily associated with mitosis and cell cycle progression with significance of p=ê−40.

In one embodiment, drugs that act as inhibitors of estrogen synthesis by functions mediated via actions directed at the Aro1 gene product can be used for the prevention of hepatocellular carcinoma in Caucasian human males by their ability to modulate the activity of the Aro1 LFNR and thereby the cell cycle-mitosis network that involves the key genes that modulate cell proliferation.

In one embodiment, the present invention provides for the prevention of hepatocellular carcinoma (HCC) development by administration of an aromatase inhibitor to high-risk Caucasian males that have the disease of chronic viral hepatitis with or without progression to cirrhosis.

In another embodiment the present invention provides that inhibition of Aro1 activity may be achieved by therapy that employs a single aromatase inhibitor or a combination of aromatase inhibitors. Such aromatase inhibitors can be selected from commercially available non-steroidal and reversible aromatase inhibitors such as Anastrozole, or from commercially available irreversible steroidal inhibitor that forms a permanent and deactivating bond with the aromatase enzyme, such as Exemestane.

By “aromatase inhibitors”, they are to be understood as substances that inhibit the enzyme aromatase (estrogen synthetase), which is responsible for converting androgens to estrogens. Aromatase inhibitors may have a non-steroidal or a steroidal chemical structure. According to the present invention, both non-steroidal aromatase inhibitors and steroidal aromatase inhibitors can be used.

The in vitro inhibition of aromatase activity can be demonstrated, for example, using described methods [J. Biol. Chem. 249, 5364 (1974) or in J. Enzyme Inhib. 4, 169 (1990)].

In vivo aromatase inhibition can be determined, for example, by the following method [See J. Enzyme Inhib. 4, 179 (1990)] wherein androstenedione (30 mg/kg subcutaneously) is administered on its own or together with an aromatase inhibitor (orally or subcutaneously) to sexually immature female rats for a period of 4 days. After the fourth administration, the rats are sacrificed and the uteri are isolated and weighed. The aromatase inhibition is determined by the extent to which the hypertrophy of the uterus induced by the administration of androstenedione alone is suppressed or reduced by the simultaneous administration of the aromatase inhibitor.

The third-generation aromatase inhibitors letrozole and anastrozole are potent and do not inhibit related enzymes. They are well tolerated and apart from their effects on estrogen metabolism their use is not associated with important side effects. Although aromatase inhibition by anastrozole and letrozole can be 100% in women, administration of these inhibitors to men does not suppress plasma estradiol levels completely. In men third-generation aromatase inhibitors decrease the mean plasma estradiol/testosterone ratio by 77%. This relates to the high plasma concentrations of testosterone, a major precursor for estradiol synthesis in adult men. Aromatase activity is high in the testes and the molar ratio of testosterone to letrozole is much higher in the testes compared with adipose and muscle tissue. When testicular testosterone and estradiol synthesis are suppressed and testosterone is administered exogenously in combination with letrozole, however, the estradiol/testosterone ratio is suppressed by 81%, which is only marginally different from the suppression of this ratio in intact men after treatment with letrozole. This incomplete suppression may be regarded as advantageous for it prevents excessive reduction of estrogen levels in men and negates possible side effects. [See W de Ronde and F H de Jong, Aromatase inhibitors in men: effects and therapeutic options. Reprod Biol Endocrinol. 2011; 9: 93. Published online 2011 Jun. 21. doi:10.1186/1477-7827-9-93 PMCID:PMC3143915; and Mauras N, O'Brien K O, Klein K O, Hayes V. Estrogen suppression in males: metabolic effects. J Clin Endocrinol Metab. 2000 July; 85(7):2370-7].

The invention also provides for the use of one or more daily doses of an aromatase inhibitor(s) either alone or in combination with a plurality of daily doses of other pharmaceutical agents.

The invention also provides for the use of one or more daily doses of at least one aromatase inhibitor in amounts thought to be potentially effective in preventing HCC

Another aspect of the invention comprises the use of an aromatase inhibitor(s) in the preparation of a medicament for use as a preventative of HCC in high-risk Caucasian males.

While one aromatase inhibitor may be preferred for use in the present invention, combinations of aromatase inhibitors may be used especially those aromatase inhibitors having different half-lives. The aromatase inhibitor can be selected from aromatase inhibitors having a half-life of about 8 hours to about 4 days, or from aromatase inhibitors having a half-life of about 2 days in the target patient population.

The aromatase inhibitors that have been found to be most useful of the commercially available forms are those in oral form. This form offers clear advantages over other forms, including convenience and patient compliance. In one embodiment, the aromatase inhibitors of the present invention include all those that are currently commercially available, including anastrozole, letrozole, vorozole and exemestane.

The daily doses required for the present invention depend on the type of aromatase inhibitor that is used. Some inhibitors are more active than others and, therefore, lower amounts of the former inhibitors may be used.

In one embodiment, the aromatase inhibitor is administered in a daily dose of from about 0.01 mg to about 500 mg. In another embodiment, the aromatase inhibitor is administered in a daily dose of from about 0.1 mg to about 50 mg. In another embodiment, the aromatase inhibitor is administered in a daily dose of from about 1 mg to about 10 mg.

In specific examples, when the aromatase inhibitor is letrozole, it may be administered in a daily dose of from about 2.5 mg to about 10 mg. When the aromatase inhibitor is anastrozole, it may be administered in a daily dose of from about 1 mg to about 30 mg. When the aromatase inhibitor is vorozole, the daily dose may be from about 5 to about 100 mg. Exemestane may be administered in a daily dose of about 1 mg to about 200 mg.

There is a scientific basis supporting the possibility that aromatase inhibitors can be used as prevention agents for hepatocellular carcinoma even though no such studies have been published in that regard. The logic of such a possibility is not obvious because it has been reported that once hepatocellular carcinoma has developed, it is not responsive to positive or negative hormone therapy. A series of papers have reporter that hormones or hormone inhibitors are not effective therapeutic agents for HCC. See, for example:
a) Massimo Di Maio, Bruno Daniele, Sandro Pignata, Ciro Gallo, Ermelinda De Maio, Alessandro Morabito, Maria Carmela Piccirillo, and Francesco Perrone. Is human hepatocellular carcinoma a hormone-responsive tumor? World J Gastroenterol. 14.1682-1689 (2008);
b) Gallo C, De Maio E, Di Maio M, Signoriello G, Daniele B, Pignata S, Annunziata A, Perrone F. Tamoxifen is not effective in good prognosis patients with hepatocellular carcinoma. BMC Cancer 6:196 (2006);
c) Nowak A K, Stockler M R, Chow P K, Findlay M. Use of tamoxifen in advanced-stage hepatocellular carcinoma. A systematic review. Cancer 103:1408-1414 (2005); and
d) Llovet J M, Bruix J. Systematic review of randomized trials for unresectable hepatocellular carcinoma: Chemoembolization improves survival. Hepatology 37:429-442 (2003)].

Nevertheless, the present invention provides unique insights concerning the following evidence as the scientific basis for the assertion that aromatase inhibitors have the potential to serve a prevention agents for hepatocellular carcinoma in high-risk Caucasian human males, namely those patients that have the disease of chronic viral hepatitis with or without associated cirrhosis:

1) The liver cell cycle-mitosis network in Caucasian human males is linked to a significant GWAS SNP for Aro1 as described herein.
2) Estrogens can promote hepatocyte proliferation via effects on the cell cycle:
a) Francavilla A, Eagon P K, DiLeo A, Polimeno L, Panella C, Aquilino A M, Ingrosso M, Van Thiel D H, Starzl T E. Sex hormone-related functions in regenerating male rat liver. Gastroenterology 91:1263-70 (1986).
b) Francavilla, J. S. Gavaler, L. Makowka, M. Barone, V. Mazzaferro, G. Ambrosino, S. Iwatsuki, F. W. Guglielmil. A. Dileo, A. Balestrazzil, D. H. van Thiel, T. E. Starzl, Estradiol and Testosterone Levels in Patients Undergoing Partial HepatectomyA Possible Signal for Hepatic Regeneration? Dig Dis Sci. 1989 June; 34(6): 818-822.
   3) Cell cycle factors can modulate sex hormone synthesis:
a) L K Mullany, E A Hanse, A Romano, C H Blomquist, J Ian Mason, B Delvoux, C Anttila, and J H Albrecht, Cyclin D1 regulates hepatic estrogen and androgen metabolism, Am J Physiol Gastrointest Liver Physiol. 2010 June; 298(6): G884-G895.Published online 2010 Mar. 25. doi:10.1152/ajpgi.00471.2009 PMCID: PMC2907223.
   4) Anti-estrogens can antagonize estrogen-induced hepatocyte proliferation via modulation of the expression of cell cycle genes and their functions:
a) A Francavilla, L Polimeno, A DiLeo, M Barone, P Ove, M Coetzee, P Eagon, L Makowka, G Ambrosino, V Mazzaferro, and T E. Starzl, The Effect of Estrogen and Tamoxifen on Hepatocyte Proliferation in Vivo and in Vitro. Hepatology 9: 614-620, 1989.
   5) Estrogens can induce chromosomal instability and aneuploidy and influence epigenetic mechanisms associated with carcinogenesis:
a) Parry, et. al., Detection and characterization of mechanisms of action of aneugenic chemicals. Mutagenesis (2002) 17 (6): 509-521.doi: 10.1093/mutage/17.6.509.
b) M Mann, V Cortez, and R K Vadlamudi, Epigenetics of Estrogen Receptor Signaling: Role in Hormonal Cancer Progression and Therapy, Cancers (Basel). 2011 Mar. 29; 3(3): 1691-1707.doi: 10.3390/cancers3021691.
c) G S Prins, Estrogen Imprinting: When Your Epigenetic Memories Come Back to Haunt You Endocrinology Dec. 1, 2008 vol. 149 no. 12 5919-5921.
d) S Dedeurwaerder, D Fumagalli, F Fuks. Unraveling the epigenomic dimension of breast cancers, Curr Opin Oncol. 2011 November; 23(6):559-65.
   6) Chronic liver disease in males that precedes the development of HCC is associated with elevated estrogen levels that can facilitate hepatocyte proliferation:
a) Yoshitsugu M, Ihori M, Endocrine disturbances in liver cirrhosis—focused on sex hormones. Nihon Rinsho. 55:3002-6 (1997).
b) Villa E, Dugani A, Moles A, Camellini L, Grottola A, Buttafoco P, Merighi A, Ferretti I, Esposito P, Miglioli L, Bagni A, Troisi R De Hemptinne B, Praet M, Callea F, Manenti F. Variant liver estrogen receptor transcripts already occur at an early stage of chronic liver disease, Hepatology 27:983-8 (1998).
   7) Cell cycle gene dysfunctions contribute to hepatocytes transformation by altering cell cycle control as demonstrated in a transgenic model of HCC and in human dysplasia that leads to HCC using biopsy specimens:
a) V R Mas, D G Maluf, K J Archer, K Yanej, X Kong, L Kulik, C E Freise, K M Olthoff R M Ghobrial, P McIver, R Fisher, Genes involved in Viral Carcinogenesis and Tumor Initiation in Hepatitis C Virus-Induced Hepatocellular Carcinoma, Mol Med. 15: 85-94 (2009).
b) Hunecke D, Spanel R, Langer F, Nam S W, Borlak J, MYC-regulated genes involved in liver cell dysplasia identified in a transgenic model of liver cancer J Pathol. 2012 May 31. doi: 10.1002/path.4059. (Epub ahead of print).
   8) Estrogens have been classified as human carcinogens:
a) R Nelson Steroidal oestrogens added to list of known human carcinogens. Lancet, 360: 2053 (2002).
b) Yu M C, Yuan J M. Environmental factors and risk for hepatocellular carcinoma. Gastroenterology 127(5 Suppl 1):572-8 (2004).

Another embodiment of this invention is the series of methods, processes, and platforms described herein to identify the Aro1 GWAS SNPs in Caucasian male liver for the cell cycle-mitosis network. These can be replicated to identify comparable cell cycle-mitosis networks and their regulatory GWAS SNPs in other normal human tissues with potential sex and race specificities (see Section 9 that follows). In another embodiment, these involve developing additional microarray-based databases for large human populations and entering them into GeneNetwork or a comparable bioinformatics analytical tool. The data set can then be analyzed by the methods herein to search the expression dataset for those genes whose expression covaries with Cdc20 or associated cell cycle or mitosis gene as described above using all the aforementioned aspects of the MCV process and the LFNR platform.

Another embodiment of this invention is the series of methods, processes, and platforms described herein to identify the Aro1 GWAS SNPs for the cell cycle-mitosis network in Caucasian male livers that are at high-risk to develop HCC namely patients that have the disease of chronic viral hepatitis with or without progression to cirrhosis. These can be replicated to identify comparable cell cycle-mitosis networks and their regulatory GWAS SNPs in other normal human tissues that have a proclivity to undergo malignant transformation and cancer development with potential sex and race specificities. In one embodiment, these involve developing additional microarray-based databases for large human populations and entering them into GeneNetwork or a comparable bioinformatics analytical tool. The data set can then be analyzed by the methods herein to search the expression dataset for those genes whose expression covaries with Cdc20 or associated cell cycle or mitosis gene as describe above using all the aforementioned aspects of the MCV process and the LFNR platform.

Patients with chronic viral hepatitis with or without associated cirrhosis are at high-risk for the development of HCC as documented by the following literature. There are approximately 170 million new cases of hepatitis C and approximately 350 million new cases of hepatitis B annually for a total of approximately 529 million new cases of viral hepatitis annually. Of these up to 90% can convert to chronic viral hepatitis. The annual conversion rate for patients that have chronic viral hepatitis to convert to hepatocellular carcinoma is between 3 to 8% depending on the type of virus that caused the chronic viral hepatitis and other patient-specific disease parameters. There are also a total of approximately 450,000 new cases of male HCC annually and approximately 700,000 new cases annually of all HCCs. 80% of all HCCs that develop worldwide derive from chronic hepatitis induced by infection with either hepatitis B or hepatitis C. [Walsh K, Alexander G J M, Update on chronic viral hepatitis, Postgrad Med J, 77:498-505, 2001; Nguyen V T, Law M G, Dore G J, Hepatitis B-related hepatocellular carcinoma: epidemiological characteristics and disease burden. J Viral Hepat. 16:453-463, 2009; and Kew M C, Epidemiology of chronic hepatitis B virus infection, hepatocellular carcinoma, and hepatitis B virus-induced hepatocellular carcinoma, Pathol Biol (Paris).58:273-277, 2010].

Section 9: Translate the Cell Cycle-Mitosis Network and its LFNRs from Non-Human Animals to Humans and to Use the Derived Human Data to Define Prevention Drug Targets and Prevention Drugs for Multiple Types of Cancer.

In another embodiment of the invention, the methods, processes, and platforms described herein above are used to translate the evidence obtained in non-human animals into human tissue datasets for use to identify and characterize the cell cycle-mitosis network for specific human tissues that have a predilection to convert into a specific type of cancer with specificity for race, sex, ethnicity, geography, age, and other identifiable population characteristics.

In certain embodiments, the datasets are then evaluated using GeneNetwork or comparable bioinformatics tools using approaches described herein above.

Another embodiment of this invention provides for the methods, processes, and platforms describe herein above to generate data regarding the cell cycle-mitosis network of specific human tissues that have a predilection to convert into a specific type of cancer with specificity for race, sex, ethnicity, geography, age, and other identifiable population characteristics in order to define LFNRs for the cell cycle-mitosis network that can serve as targets for drugs with the potential to prevent that cancer types of interest.

Another embodiment of this invention provides for the methods, processes, and platforms describe herein above to generate data regarding the cell cycle-mitosis network of specific human tissues that have a predilection to convert into a specific type of cancer with specificity for race, sex, ethnicity, geography, age, and other identifiable population characteristics and to use LFNR targets for the cell cycle-mitosis network to develop drugs with the potential to prevent the cancer types of interest.

Thereby, it will be possible to develop new systems genetics-based personalized cancer prevention drugs for a wide spectrum of human cancer types.

Section 10: Translate the Cell Cycle-Mitosis Network and its LFNRs from Non-Human Animals to Humans and to Use the Derived Human Data to Define Therapy Drug Targets and Therapy Drugs for Multiple Types of Cancer.

In another embodiment of the invention, the methods, processes, and platforms described herein above are used to translate the evidence obtained in non-human animals into human cancer (tumor) datasets for use to identify and characterize the cell cycle-mitosis network for specific human cancers with specificity for race, sex, ethnicity, geography, age, and other identifiable population characteristics.

In certain embodiments, tissues from specific cancer types with race and sex specificity are to be obtained from large patient populations for the purpose of developing microarray-based gene expression datasets for each type of specific cancer and its subtypes. The datasets are then evaluated using GeneNetwork or comparable bioinformatics tools using approaches described herein above.

Another embodiment of this invention provides for the methods, processes, and platforms describe herein above to generate data regarding the cell cycle-mitosis network of the specific human cancers (tumor tissue) with specificity for race, sex, ethnicity, geography, age, and other identifiable population characteristics in order to define LFNRs for the cell cycle-mitosis network for specific cancers as drug targets for the cancer type of interest

er for aromatase inhibitor treatment in a human Caucasian male subject. In another networks to develop actual drugs with the potential to treat the cancer types of interest.

Thereby, it will be possible to develop new systems genetics-based personalized cancer therapy drugs for a wide spectrum of human cancer types.

1.-122. (canceled)

123. A multiple criteria process to validate a systems genetics network of genes that have a common function comprising:
(a) selecting a candidate network comprising covariate expressed genes that have a common function identified as associated with a gene of interest in a test population; and
(b) determining if the identified candidate systems genetics network show covariate expression of network genes in a population data set selected from the group consisting of:
i. two or more tissue or cell types;
ii. two or more data sets developed by different laboratories or different investigators or both;
iii. two or more different microarray platforms;
iv. two or more different animal species or strains; and
v. two or more different microarray data normalization systems;
wherein the identified candidate systems genetics network is validated if it is determined that the network of covariate expressed genes with a common function are identified as having correlation coefficients greater than or equal to 0.5 or higher in two or more of the test populations.

124. The process of claim 123, wherein the process further compromises the step (c) determining that the identified candidate systems genetics network has one or more suggestive or significant eQTL in one or more test populations by using one or more systems genetics bioinformatics tool and wherein the eQTLs for the candidate network as defined in step (c) varies in different species, strains, tissues, cell types and sexes.

125. The process of claim 124, wherein the process further comprises the step (d) determining that the identified candidate systems genetics network exists substantially more in tissues or cells that physiologically express the function of the identified network than in tissues or cells that do not express the function or express the function to a lesser degree or extent.

126. The process of claim 125, wherein the candidate network is a cell cycle-mitosis network that consist of sets of genes that control the G1, S, G2 or M phases of the cell cycle and show covariate expression with a cell cycle gene of interest.

127. A method for identifying the linked function network regulator (LFNR) of a systems genetics network of interest comprising:
(a) screening a plurality of eQTLs identified in claim 2 for candidate eQTGs associated with the eQTLs for the network of interest; and
(b) identifying a linked function shared by the candidate eQTGs in each population; wherein the eQTGs identified as having a linked function are designated as candidate linked function network regulators (LFNRs) for the network.

128. The process of claim 127, wherein the linked function network regulator is a gene product with a function linked with the network regulated by the linked function network regulator.

129. The process of claim 128, wherein the candidate eQTGs associated with the eQTLs of the network of interest in various populations are analyzed using bioinformatics tools and wherein the eQTLs for the network of interest contain a distinct composition of genes with a linked function in a plurality of populations selected from the group consisting of species, strains, tissues, cell types and sexes.

130. The process of claim 129, comprising the further step of validating the candidate eQTGs associated with eQTLs for a specific network by identifying the eQTGs in multiple populations and wherein all cis candidate eQTGs are analyzed for each of populations to identify a linked function shared by the candidate eQTGs in each population.

131. The process of claim 130, wherein a subset of the candidate cis eQTGs is identified as having a linked function that is shared with each population and wherein the subset genes identified are designated as the linked function network regulators for the network.

132. The process of claim 131, wherein a subset of the candidate trans eQTGs is identified as having a linked function that is shared with each population and wherein the subset genes identified are designated as the linked function network regulators for the network.

133. The process of claim 132, wherein the candidate network is a cell cycle-mitosis network.

134. An article comprising a data set of genes that comprise a network that share a common cell cycle and/or mitosis function whose expression is covariate and whose function is regulated by a linked function network regulator.

135. The article of claim 134, wherein the covariate expressed genes have a correlation coefficient >0.5 in a population selected from the group consisting of different species, strains, sexes and tissues.

136. The article of claim 135, wherein QTLs are identified for the cell cycle-mitosis network in a plurality of tissues and cells of different species, strains, sexes and wherein the QTLs are used to identify a linked function network regulator for the cell cycle-mitosis network in each situation.

137. The article of claim 136, wherein the characteristics of the cell cycle-mitosis network and the LFNRs for the network in non-human animals provides a model for translation to humans as new drug targets for the prevention, amelioration or treatment of cancer and other human diseases.

138. A method for identifying human candidate cell cycle-mitosis networks and their linked function network regulators, the method comprising the steps of:
(a) selecting a human gene expression data set of interest representing a population of tissues or cells with significant genetic variation and
(b) analyzing the data set using a candidate gene of interest to identify cell cycle and/or mitosis genes whose expression is covariate;
(c) selecting a set of genes having cell cycle and/or mitosis function and designating that set of genes as a network.

139. A method of claim 138, wherein the human populations of one or more types of cells and/or tissues are selected based on one or more characteristic selected from the group consisting of race, sex, ethnicity, geography, age, and other identifiable population characteristics.

140. A method of claim 139, wherein the data sets used to screen for the cell cycle-mitosis network and for GWAS SNPs employ gene expression information obtained from whole genome expression arrays or from specially designed sets of gene expression arrays that related to the cell cycle and/or cancer.

141. The method of claim 140, further comprising identifying GWAS SNPs for the selected cell cycle-mitosis network genes in a plurality of human tissue or cell populations and wherein the GWAS SNP candidates having the highest significance and having a cell cycle or mitosis function are designated as candidate LNFRs.

142. The method of claim 141, wherein the GWAS SNPs have a significance of 5.0−log P or greater.
